(19) United States
(12) Patent Application Publication
(10) Pub. No.: US 2018/0125474 A1
(43) Pub. Date: May 10, 2018

(54) AUTOMATED HAND-HELD PERCUSSIVE MEDICAL DEVICE AND SYSTEMS, KITS, AND METHODS FOR USE THEREWITH

(71) Applicant: Tenjin LLC, Brazoria, TX (US)

(72) Inventors: Christopher P. Dougherty, Rogers, AR (US); Robert A. Van Wyk, St. Pete Beach, FL (US); Gary R. Heisler, Brazoria, TX (US)

(73) Assignee: Tenjin LLC, Brazoria, TX (US)

(21) Appl. No.: 15/805,654

(22) Filed: Nov. 7, 2017

Related U.S. Application Data
(60) Provisional application No. 62/497,066, filed on Nov. 7, 2016.

(51) Int. Cl. A61B 17/04; A61B 17/00

(52) U.S. Cl. CPC A61B 17/0401; A61B 17/00234; A61B 2017/0438; A61B 2017/0403; A61B 2017/0427; A61B 2017/0409

(57) ABSTRACT

A number of surgical procedures, particularly orthopaedic procedures, involve manual percussion, typically mallet-applied. As discussed in detail herein, such procedures can be problematic as they not only require a second set of hands but are often associated with incomplete and/or uncontrolled axial movement and/or injury to the surgeon's hands. Described herein are specialized hand-held devices, as well as systems, kits, and methods associated therewith, that avoid such problems by joining or replacing the external “mallet” with an internal “hammer” that can automate the delivery of controlled repetitive “strikes”, more particularly repeated strikes to a proximal end of a drill, driver and/or insertion device, so as to mete out pre-determined incremental movement to a distal end component, such as a sharpened socket-forming tip, an interference-plug type suture anchor, or a dilating device for modifying the cross-section of a tunnel formed in a bone. The present invention has particular utility in connection with ligament, tendon and joint reconstruction procedures, such as shoulder, ankle, and knee repair. The present invention is also applicable to those surgical procedures that require the production of and access to off-axis bone sockets.

### PRIORITY

This application claims the benefit of U.S. Provisional Application Ser. No. 62/497,066 filed Nov. 7, 2016, the contents of which are hereby incorporated by reference in its entirety.

### TECHNICAL FIELD OF THE INVENTION

The present invention relates generally to the field of endoscopic and arthroscopic surgery and interference-plug type suture anchor systems for use therein. More particularly, the invention relates to an automated hand-held percussive device, as well as systems, kits and methods for use therewith, in which conventional manual percussion, typically mallet-applied, is joined with or replaced by the automated delivery of controlled repetitive “strikes”, more particularly repeated strikes to a proximal end of a driver and/or insertion device, so as to mete out pre-determined incremental movement to a distal end component, examples of which include a sharpened socket-forming tip, interference-plug type suture anchors, or square or polygonal dilating devices for modifying the cross-section of a tunnel formed in a bone. The present invention has particular applicability to ligament, tendon and joint reconstruction procedures, such as shoulder, ankle, and knee repair. The present invention also has particular applicability to those surgical procedures that require the production of and access to off-axis bone sockets.

### BACKGROUND OF THE INVENTION

Push-in implants and interference-plug type anchors for affixing suture(s) or tissue(s) to bone are well known in the art. Illustrative examples of these implants include the Knotless Push-In Anchors by Parcus Medical (Sarasota, Fla.) and the PushLock® and SutureTak® Suture anchors by Arthrex, Inc. (Naples, Fla.). In conventional practice, the implant is mounted to the distal end of a driver, which the surgeon positions at a prepared socket in which the implant is to be placed. While the surgeon maintains the placement of the distal end of the driver and further positions an endoscope so as to confirm proper positioning of the driver, the proximal end of the driver is struck repeatedly with a mallet to drive the implant into the socket. This practice has a number of disadvantages. First, it requires a second set of hands and thus cannot be performed by a surgeon without assistance. Second, the mallet frequently misses the proximal end of the driver and painfully strikes the surgeon's hand. Third, as the force is manually applied, it is virtually impossible to deliver a consistent, controlled, metered amount of force. Moreover, as the surgeon does not directly control the delivery of force, he likewise cannot directly control the depth to which the implant is placed. As such, the implant may frequently be over-driven into the socket. This over insertion may result in sub-optimal (excessive) tension of the associated sutures

In view of these distinct disadvantages, there is a need in the art for an automated hand-held placement system for push-in implants and the like that gives the surgeon improved control over the placement process and that spares the surgeon's hands.

### SUMMARY OF THE INVENTION

A primary objective of the present invention is to provide improved means and methods for attaching soft tissues (i.e., “grafts”) to bone in situ. The present invention is particularly concerned with those procedures that involve the step of driving an interference-plug type anchor into a prepared socket, a step conventionally performed with the use of an external mallet that, as noted above, can be quite problematic. Thus, it is an object of the present invention to provide means and mechanisms to address the problems in the prior art by providing an automated, hand-held percussive device capable of incrementally advancing a push-in implant into a prepared socket so as to provide the surgeon with total control over the placement process. Specifically, the percussive medical device of the present invention allows the surgeon to single-handedly both position and drive the implant into the socket while ensuring proper alignment and placement using an endoscope held in the surgeon's other hand. The device may also be used to form sockets or other small circular or square holes, such as those used for microfracture treatment of articular cartilage lesions.

It is a further object of the present invention to provide a hand-held percussive device in which energy supplied by a surgeon, using a trigger or other input means, is converted to potential energy in an elastic element, and thereafter to kinetic energy through acceleration of a weight, which, in turn, is applied percussively to a proximal portion of an elongate driver assembly or insertion device assembled to the distal end of the percussive device and then on to the distal end of said driver assembly or insertion device so as to incrementally advance a sharpened distal portion into bone or to place an implant in a prepared socket.

The hand-held percussive devices of the present invention convert energy input to the device by the surgeon, via a suitable mechanical input means, to potential energy stored in a compression spring by compression of the spring. As the compressed spring is released, the energy stored therein is converted to kinetic energy so as to propel a weight in the distal direction. Near the end of its distal travel, the weight strikes the proximal end of a distally extending, axially translatable, metallic element such that the kinetic energy of the weight is percussively transferred to this metallic element. In the context of the present invention, the metallic element is preferably an elongate driver device and/or insertion assembly or a component thereof. In this context, the distal end of the elongate distal device may be sharpened, configured for the penetration of a boney surface as when forming a socket for implant placement or performing a microfracture procedure.

Alternatively, the distal end of the elongate distal device may be configured for the removable placement thereon of an interference plug-type implant, such that percussive energy supplied by the percussive device of the instant invention to the distal end of the elongate distal device incrementally inserts the implant into a prepared socket. In certain embodiments, the implant may be cannulated, with the distal-most portion of the elongate distal device protruding beyond the implant. The protruding portion may optionally be sharpened to penetrate bone such that a tunnel may be formed thereby simultaneously with the placement of the implant. In other embodiments, the distal portion of the elongate distal device may be configured for dilating a previously drilled bone tunnel of the type routinely formed in procedures for ligament repair in a knee. Regardless of the configuration of the distal end of the elongate distal device, percussive energy transferred to the elongate distal device and distal end thereof, under direct control of the surgeon, incrementally advances the distal end into a boney surface, either penetrating the boney surface to form a feature therein, or to incrementally insert an implant to a predetermined depth in a previously formed socket.

Manual percussive devices of the present invention have certain essential elements and subassemblies that together enable the conversion of energy input by the surgeon via a movable element into percussive force applied to a distal element. For example:

A case must enclose the various mechanical elements and maintain proper positions and alignments thereof. For instance, the case must maintain axial alignment of the spring and weight (together a first subassembly) with the elongate distal element. Also, the case must provide pivoting and alignment features for the linkage elements (together a second subassembly) that together transfer force supplied by the surgeon to axial movement of the weight so as to compress the spring, and to then subsequently release the weight when a predetermined level of compression is achieved. In a preferred embodiment, the case is formed of a suitable polymeric material, as an assembly of two laterally opposed elements that are joined to form the single unit. The joining may be by mechanical fasteners, ultrasonic welding, solvent/adhesive bonding, or any other suitable method. In exemplary embodiments described in detail herein, the case is configured with a pistol grip. Other embodiments, such as radial symmetry are contemplated by the present invention.

In the preferred embodiments described in detail hereinbelow, the energy is input to the device by the surgeon using a rotationally mounted trigger that protrudes from the handle portion of the case, a single pull of the trigger that both compresses the spring through proximal movement of the weight, and releases the weight when a predetermined degree of compression is achieved. However, other configurations are contemplated and thus the present invention is not limited to the disclosed embodiments. For example, the present invention contemplates embodiments in which energy is input to the device by the surgeon via a linearly acting trigger, or more generally, by any translatable element that moves relative to the case due to force applied by the surgeon so as to move a weight and compress a spring for the purpose of applying percussive force to a distally extending element.

As noted above, the present invention contemplates a first assembly composed of a compression spring and weight that together are slidably positioned within the case in a manner that permits the weight to be moved proximally to compress the spring and thereby store energy input by the surgeon as potential energy in the spring. In a preferred embodiment, a feature on the weight engages with the above-described “linkage subassembly” such that motion of the trigger is converted to axial movement of the weight. The weight engagement feature also provides automatic disengagement from the linkage subassembly when a predetermined distance of proximal travel of the weight is achieved, such that potential energy stored in the spring is converted to kinetic energy as the weight is accelerated distally. In the illustrative examples described in detail below, this engagement feature is a tangential groove formed in the outer surface of the weight near the proximal end of the weight. However, the present invention is not limited thereto and thus other analogous engagement features are may be utilized. For instance, one or more teeth may be formed on the circumferential surface of the weight, the teeth configured to engage with teeth formed on the periphery of a rotational element (e.g., a gear) such that rotational motion from the rotational element causes proximal axial motion of the weight, and disengagement at an end of the threaded portion of the rotational element. Herein, any engagement feature (or set of features) that provide for proximal movement to a predetermined distance followed by automatic disengagement is deemed to fall within the scope of the present invention. In examples herein, a compression spring is disclosed as a preferred means for storing the potential energy of the weight. However, other equivalent embodiments are contemplated; for example, the energy storage means may take the form of an extension spring or a pneumatic element. These and any other suitable energy storage means are considered to fall within the scope of the present invention.

In the illustrative examples below, the linkage subassembly is a collection of elongate elements and springs configured to transfer motion of an actuating mechanism, such as a trigger element, to the weight/spring subassembly so as to compress the spring, to automatically disengage from the weight to allow distal acceleration of the weight, and to then return the trigger element and linkage assembly to its original state in preparation for repetition of the process. However, the precise configuration of elements of the linkage subassembly depicted in the Examples below is intended for illustration purposes only. The various linkage assembly components may be modified in shape, size, and joining methods to form other linkage subassemblies that perform the equivalent function without departing from the principles of the present invention. Alternatively, the linkage assembly may use rotational elements, such as gears and gear segments, to convert motion of the trigger device to proximal motion of the weight. So long as a linkage subassembly performs this critical function and includes a means for automatically engaging and disengaging from the weight/spring subassembly, it is considered to fall within the scope of this invention.

The functional distal element may be part of a distal device or assembly, for example part of an elongate driver device or implant insertion assembly. The distal device or assembly may have an elongate element wherein the proximal portion is axially slidably assembled to the percussive case and positioned such that when the proximal end is struck by the weight, the elongate element travels distally relative to the case, and the distal end of the elongate element is configured for forming features in bone or for the placement of an implant. The distal assembly may optionally include a proximal spring to maintain the distal assembly at the proximal end of its travel when in its free state, in preparation for use. When configured in this manner, the distal assembly is irremovably assembled to the other subassemblies of the device. Forming a socket in a boney surface and subsequently placing an interference-type plug device in that socket can require two devices of the present invention, when configured as described above: one to form the socket and the other to place the implant. In other configurations, the distal assembly may be removably assembled to the other subassemblies of the percussive device such that a single device with interchangeable distal assemblies may be used to form a socket and place an anchor therein. In yet other contemplated embodiments, the distal assembly may have a first proximal portion that is irremovably mounted to the other subassemblies, and a distal portion that is removably mounted to the proximal portion. In these embodiments, forming a socket and subsequently placing an anchor in the socket may be accomplished using two interchangeable distal portions of the distal assembly, the first configured for forming the socket and the second for placing the implant. Distal assemblies of percussive devices of the present invention may be rigidly linear or may have a distal portion formed of a plurality of discreet elements configured for transmitting percussive force to a distal-most element configured for forming a socket or for placing an implant therein. When used with an external tubular guide, the distal portion of which is angularly offset from the proximal handle portion, these embodiments are able to form features in bone and place implants in locations that are not accessible when using a rigidly linear instrument. Accordingly, the present invention contemplates kits including multiple devices and components useable together for a wide array of surgical purposes.

These and other aspects are accomplished in the invention herein described, directed to a hand-held automated percussive medical device to position and drive an anchor of a graft to bone. Further objects and features of the invention will become more fully apparent when the following detailed description is read in conjunction with the accompanying figures and examples. However, it is to be understood that both the foregoing summary of the invention and the following detailed description are of a preferred embodiment, and not restrictive of the invention or other alternate embodiments of the invention. In particular, while the invention is described herein with reference to a number of specific embodiments, it will be appreciated that the description is illustrative of the invention and is not constructed as limiting of the invention.

### BRIEF DESCRIPTION OF THE FIGURES

Various aspects and applications of the present invention will become apparent to the skilled artisan upon consideration of the brief description of figures and the detailed description of the present invention and its preferred embodiments that follows:

FIG. 1 is a perspective view of a distal assembly for a hand-held percussive medical device of the present invention.

FIG. 2 is a side elevational view of the objects of FIG. 1.

FIG. 3 is a plan view of a weight and spring assembly for a hand-held percussive medical device of the present invention.

FIG. 4 is a perspective view of the objects of FIG. 3.

FIG. 5 is an expanded sectional view of the objects of FIG. 3 at location A-A.

FIG. 6 is a side elevational view of a linkage assembly for a hand-held percussive medical device of the present invention.

FIG. 7 is a perspective view of the objects of FIG. 6.

FIG. 8 is a side elevational view of a trigger for a hand-held percussive medical device of the present invention.

FIG. 9 is a perspective view of the objects of FIG. 8.

FIG. 10 is a distal perspective view of a first case half for a hand-held percussive medical device of the present invention.

FIG. 11 is an axial view of the objects of FIG. 10.

FIG. 12 is a side elevational view of the objects of FIG. 10.

FIG. 13 a proximal perspective view of the objects of FIG. 10.

FIG. 14 is a side elevational view of the assembled elements for a hand-held percussive medical device of the present invention with a second case half removed so as to show internal construction details with the device in a first condition.

FIG. 15 is an expanded view of the objects of FIG. 14 at location A.

FIG. 16 is a perspective view of the objects of FIG. 14.

FIG. 17 is a side elevational view of the assembled elements for a hand-held percussive medical device of the present invention with a second case half removed so as to show internal construction details with the device in a second condition.

FIG. 18 is an expanded view of the objects of FIG. 17 at location A.

FIG. 19 is a perspective view of the objects of FIG. 17.

FIG. 20 is a perspective view of a hand-held percussive medical device of the present invention.

FIG. 21 is a side elevational view of the objects of FIG. 20.

FIG. 22 is an expanded view of the distal portion of the objects of FIG. 21 at location A.

FIG. 23 is an expanded view of the distal portion of an alternate embodiment hand-held percussive device of the present invention.

FIG. 24 is an expanded view of the distal portion of an alternate embodiment hand-held percussive device of the present invention.

FIG. 25 is an expanded view of the distal portion of an alternate embodiment hand-held percussive device of the present invention.

FIG. 26 is a side elevational view of an alternate embodiment hand-held percussive device of the present invention.

FIG. 27 is a perspective view of the objects of FIG. 26.

FIG. 28 depicts the alternate embodiment percussive device of FIG. 26 with a portion of the enclosure removed to show the internal construction.

FIG. 29A is a perspective view of a prior art driver device and an implant positioned for removable mounting thereon.

FIG. 29B is an expanded view of the objects of FIG. 29 at location A.

FIG. 30A is a plan view depicting the prior art driver device and implant of FIGS. 29A and 29B with the implant removably mounted to the distal end of the driver device in preparation for use.

FIG. 30B is a perspective view of the objects of FIG. 31.

FIG. 31 is a perspective view of an alternate embodiment of the present invention configured for the off-axis placement of push-in implants.

FIG. 32 is an expanded view of the distal portion of the objects of FIG. 31.

FIG. 33 is a side elevational view of the objects of FIG. 31.

FIG. 34 is an expanded view of the objects of FIG. 33 at location C.

FIG. 35 is an expanded view of the objects of FIG. 32 at location E.

FIG. 36 is an expanded sectional view of the objects of FIG. 33 at location A-A.

FIG. 37 is a side elevational view of an alternate embodiment of the present invention configured for the placement of off-axis sockets in a boney surface.

FIG. 38 is an expanded view of the objects of FIG. 37 at location A.

FIG. 39A is a perspective view of a guide of the present invention having an angularly offset distal portion and configured for use with the alternate embodiments of FIGS. 31 and 37.

FIG. 39B is an expanded view of the objects of FIG. 39A at location A.

FIG. 40 is a plan view of the objects of FIG. 39.

FIG. 41 is a side elevational view of the objects of FIG. 39.

FIG. 42 is a sectional view of the objects of FIG. 40 at location A-A.

FIG. 43 depicts a first step in the placement of a push-in suture anchor according to principles of the present invention in which the guide of FIG. 39 is positioned on a boney surface at a predetermined location.

FIG. 44 depicts a second step in the placement of a push-in suture anchor according to principles of the present invention in which the distal portion of the punching embodiment of FIG. 37 is inserted into the guide of FIG. 39.

FIG. 45 depicts the placement of a push-in suture anchor according to principles of the present invention in which a third step, the forming of a socket by the punching embodiment of FIG. 37, has been completed.

FIG. 46 depicts a fourth step in the placement of a push-in suture anchor according to principles of the present invention in which the distal portion of the embodiment of FIG. 31 with a push-in implant is inserted into the guide of FIG. 39.

FIG. 47 depicts the placement of a push-in suture anchor according to principles of the present invention in which a fifth step, the placement of a push-in suture anchor in a prepared socket is completed.

FIG. 48 depicts a suture anchor placed by a method of the previous invention at the completion of the process.

FIG. 49 is a perspective view of a drill with a flexible portion configured for use with the guide of FIG. 39.

FIG. 50 is a side elevational view of the objects of FIG. 49.

FIG. 51 is an expanded view of the objects of FIG. 49 at location A.

FIG. 52 is an expanded view of the objects of FIG. 50 at location B.

FIG. 53 is a plan view of the drill of FIG. 49 inserted in the guide of FIG. 39 in preparation for forming a socket in an alternate embodiment of the implant placement method previously depicted.

FIG. 54 is a side elevational view of the objects of FIG. 53.

FIG. 55 is an expanded sectional view of the distal portion of the objects of FIG. 54 at location A-A.

FIG. 56 is a side elevational view of an alternate embodiment device of the present invention.

FIG. 57 is an expanded view of the objects of FIG. 56 at location A.

FIG. 58 is a perspective view of the objects of FIG. 56.

FIG. 59 is an expanded view of the objects of FIG. 58 at location B.

### DETAILED DESCRIPTION OF THE PREFERRED EMBODIMENTS

Aspects of the present invention relate to, overlap with and/or find utility in conjunction with aspects described in the following related patents and patent applications, the entire contents of which are hereby incorporated in their entirety:
U.S. Pat. No. 9,226,817 issued Jan. 5, 2016; U.S. Pat. No. 9,566,060 issued Feb. 14, 2017; and U.S. Pat. No. 9,795,374 issued Oct. 24, 2017, all of which are entitled “Implant Placement Systems, Devices, and Methods”;
U.S. application Ser. No. 15/256,815 filed Sep. 6, 2016, entitled “Ceramic Implant Placement Systems And Superelastic Suture Retention Loops For Use Therewith”, published as US 2017/0000476 on Jan. 5, 2017, and issued as U.S. Pat. No. 9,770,240 on Sep. 26, 2017;
U.S. application Ser. No. 15/256,838 filed Sep. 6, 2016, entitled “Implant Placement Systems And One-Handed Methods For Tissue Fixation Using Same”, published as US 2016/0367357 on Dec. 22, 2016, and issued as U.S. Pat. No. 9,782,250 on Oct. 10, 2017;
U.S. application Ser. No. 15/256,945 filed Sep. 6, 2016, entitled “Multiple Implant Constructions and Fixation Methods Associated Therewith”, published as US 2016/0374795 on Dec. 29, 2016, and issued as U.S. Pat. No. 9,717,587 on Aug. 1, 2017;
U.S. application Ser. No. 14/583,232 filed Dec. 24, 2014, entitled “Percussive Surgical Devices, Systems, and Methods of Use Thereof′ and published as US 2015/0182233 on Jul. 2, 2015;
U.S. application Ser. No. 14/635,266 filed Mar. 2, 2015, entitled “Bendable and Rebendable Endoscopic Electrosurgical Device” and published as US 2015/0245868 on Sep. 3, 2015;
U.S. application Ser. No. 14/817,061 filed Aug. 3, 2015, entitled “Suture-Anchoring Implanted Medical Devices Engineered from Bioresorbable Metal Alloys”, published as US 2016/0030032 on Feb. 4, 2016, and issued as U.S. Pat. No. 9,597,069 on Mar. 21, 2017; and
U.S. application Ser. No. 15/288,509 filed Oct. 7, 2016, entitled “Powered Endoscopic Drilling Device” and published as US 2017/0100136 on Apr. 13, 2017.

Although any methods and materials similar or equivalent to those described herein can be used in the practice or testing of embodiments of the present invention, the preferred methods, devices, and materials are now described. However, before the present materials and methods are described, it is to be understood that the present invention is not limited to the particular sizes, shapes, dimensions, materials, methodologies, protocols, etc. described herein, as these may vary in accordance with routine experimentation and optimization. It is also to be understood that the terminology used in the description is for the purpose of describing the particular versions or embodiments only, and is not intended to limit the scope of the present invention which will be limited only by the appended claims. Accordingly, unless otherwise defined, all technical and scientific terms used herein have the same meaning as commonly understood by one of ordinary skill in the art to which the present invention belongs. However, in case of conflict, the present specification, including definitions below, will control.

In the context of the present invention, the following definitions apply:

The words “a”, “an” and “the” as used herein mean “at least one” unless otherwise specifically indicated. Thus, for example, reference to an “opening” is a reference to one or more openings and equivalents thereof known to those skilled in the art, and so forth.

The term “proximal” as used herein refers to that end or portion which is situated closest to the user of the device, farthest away from the target surgical site. In the context of the present invention, the proximal end of the implant system of the present invention includes the driver and handle portions.

The term “distal” as used herein refers to that end or portion situated farthest away from the user of the device, closest to the target surgical site. In the context of the present invention, the distal end of the implant systems of the present invention includes components adapted to fit within the pre-formed implant-receiving socket.

In the context of the present invention, the terms “cannula” and “cannulated” are used to generically refer to the family of rigid or flexible, typically elongate lumened surgical instruments that facilitate access across tissue to an internally located surgery site.

The terms “tube” and “tubular” are interchangeably used herein to refer to a generally round, long, hollow component having at least one central opening often referred to as a “lumen”.

The terms “lengthwise” and “axial” as used interchangeably herein to refer to the direction relating to or parallel with the longitudinal axis of a device. The term “transverse” as used herein refers to the direction lying or extending across or perpendicular to the longitudinal axis of a device.

The term “lateral” pertains to the side and, as used herein, refers to motion, movement, or materials that are situated at, proceeding from, or directed to a side of a device.

The term “medial” pertains to the middle, and as used herein, refers to motion, movement or materials that are situated in the middle, in particular situated near the median plane or the midline of the device or subset component thereof.

The term “radial” is used herein to refer to characterize movement inward and outward from a central point or shaft, e.g., thrusts radial to the center of rotation.

The present invention contemplates the use of alternative cooperating elements, in particular cooperating elements disposed within the hand-held case assembly, for automatically transmitting relative axial movement to a distal “driver” or “insertion” component from a proximal “hammer component”, such as a weight-and-spring assembly, when an actuator, such as a “trigger element”, is relatively rotated or otherwise actuated. While the invention is described herein below with respect to a rotating trigger and a spring-held weight, other cooperating elements are contemplated, examples of which include, but are not limited to, screw threads, worm gears, worm wheels, pneumatic devices, hydraulic mechanisms, magnetic assemblies, ratchet-and-pawl assemblies, and push-pull connectors.

As discussed above, when a tissue, more particularly a soft connective tissue in a joint space, becomes damaged or torn from its associated bone or cartilage, surgery is usually required to reattach the tissue or reconstruct the bone. The present invention is directed to various means and mechanisms for securing the displaced tissue to boney tissue.

As used herein, the term “tissue” refers to biological tissues, generally defined as a collection of interconnected cells that perform a similar function within an organism. Four basic types of tissue are found in the bodies of all animals, including the human body and lower multicellular organisms such as insects, including epithelium, connective tissue, muscle tissue, and nervous tissue. These tissues make up all the organs, structures and other body contents. While the present invention is not restricted to any particular soft tissue, aspects of the present invention find particular utility in the repair of connective tissues such as ligaments or tendons, particularly those of the shoulder, elbow, knee or ankle joint.

In a similar fashion, while the present invention is not restricted to any particular boney tissue, a term used herein to refer to both bones and cartilage, aspects of the present invention find particular utility in the repair or reattachment of connective tissues to the boney elements of the shoulder, elbow, wrist, hand, hip, knee or ankle joint.

When the damaged tissue is of sufficient quantity and quality, the damaged portion may simply be directly reattached to the bone from which it was torn so that healing back to the bone can take place. However, in other situations, a “graft” may be needed to stimulate regrowth and permanent attachment. In the context of the present invention, the term “graft” refers to any biological or artificial tissue being attached to the boney tissue of interest, including:
Autografts, i.e., grafts taken from one part of the body of an individual and transplanted onto another site in the same individual, e.g., ligament graft;
Isografts, i.e., grafts taken from one individual and placed on another individual of the same genetic constitution, e.g., grafts between identical twins;
Allografts, i.e., grafts taken from one individual placed on genetically non-identical member of the same species; and
Xenografts, i.e., grafts taken from one individual placed on an individual belonging to another species, e.g., animal to man.

Autografts and isografts are usually not considered as foreign and, therefore, do not elicit rejection. Allografts and xenografts are recognized as foreign by the recipient thus carry a high risk of rejection. For this reason, autographs and isografts are most preferred in the context of the present invention.

Surgical interventions such as contemplated herein generally require the boney tissue to be prepared for receiving the graft. In the context of the present invention, such preparation includes the formation of a “socket”, i.e., a hole punched or drilled into the bone into which a prosthetic device such as an implant may be received. The hand-held percussive medical device of the present invention finds particular utility both in the preparation of such a socket at a desired target location and in the placement of an interference-plug type suture anchor into the prepared socket. Some interference-plug type suture anchors known as “self punching” form the socket simultaneously with insertion of the implant, with the distal portion of the implant or of the driver forming the tunnel during insertion of the implant. Such implants may likewise be placed using hand-held percussive devices of the present invention.

While certain procedures contemplate directly attaching the graft to the bone, the more common route involves the employment of an implant or anchor specially configured to hold and/or enable attachment of the graft to the boney tissue. As used herein, the terms “implant” and “anchor” are interchangeably used herein to refer to a prosthetic device fabricated from a biocompatible and/or inert material. In the context of the present invention, examples of such “implants” include conventional and knotless anchors of the push-in and interference-fit variety.

In certain embodiments, the present invention contemplates the use of implants fabricated from either a metallic material or a suitable polymeric material, including, but not limited to, polyetheretherketone (PEEK), a polymeric composite such as, for instance, carbon fiber reinforced PEEK (PEEK CF), or of a suitable bioabsorbable material such as, for instance, polylactic acid (PLA). The present invention also contemplates the use of very small knotless anchors produced from ceramic materials using a process known as “Ceramic Injection Molding” or simply “CIM”. The tensile strength of PEEK material is typically between 10,000 and 15,000 psi. In comparison, the tensile strength of alumina is generally in excess of 200,000 psi. Furthermore, recently developed materials such as Zirconia Toughened Alumina (ZTA) by Coorstek Inc. (Golden, Colo.) have a high degree of toughness in addition to high tensile strength. These materials, being ceramic, do not have a yield point and therefore do not deform under load. The high tensile strength and the absence of yielding under load of an implant constructed of such ceramic materials allow torque to be transmitted to the implant through features that are not producible by the machining of metal or that would fail in use if formed from a polymeric material such as PEEK.

In certain embodiments, the implant may take the form of a ceramic interference plug, wherein the high elastic modulus and high strength of the ceramic materials is beneficial for small and miniature interference type anchors that are driven axially into a prepared socket. The high modulus and high strength of the materials allows the thickness of the wall between the central lumen and the outer surface to be reduced compared to interference type anchors produced from polymeric materials without reducing the compressive force which retains the one or more sutures between the outer wall of the implant and the wall of the socket.

The present invention contemplates securing a graft to a boney surface via sutures. In the context of the present invention, the term “suture” refers to a thread-like strand or fiber used to hold body tissues after surgery. Sutures of different shapes, sizes, and thread materials are known in the art and the present invention is not restricted to any particular suture type. Accordingly, in the context of the present invention, the suture may be natural or synthetic, monofilament or multifilament, braided or woven, permanent or resorbable, without departing from the spirit of the invention.

The instant invention has both human medical and veterinary applications. Accordingly, the terms “subject” and “patient” are used interchangeably herein to refer to the person or animal being treated or examined. Exemplary animals include house pets, farm animals, and zoo animals. In a preferred embodiment, the subject is a mammal, more preferably a human.

Hereinafter, the present invention is described in more detail by reference to the Figures and Examples. However, the following materials, methods, figures, and examples only illustrate aspects of the invention and are in no way intended to limit the scope of the present invention. For example, while the present invention makes specific reference to arthroscopic procedures, it is readily apparent that the teachings of the present invention may be applied to other minimally invasive procedures and are not limited to arthroscopic uses alone. As such, methods and materials similar or equivalent to those described herein can be used in the practice or testing of the present invention.

### EXAMPLES

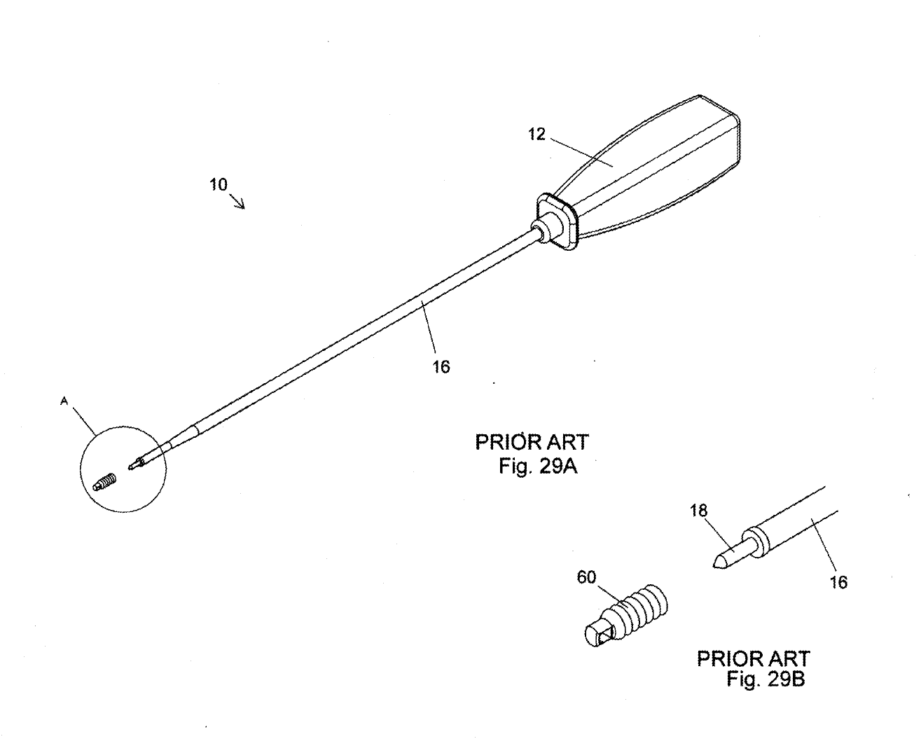

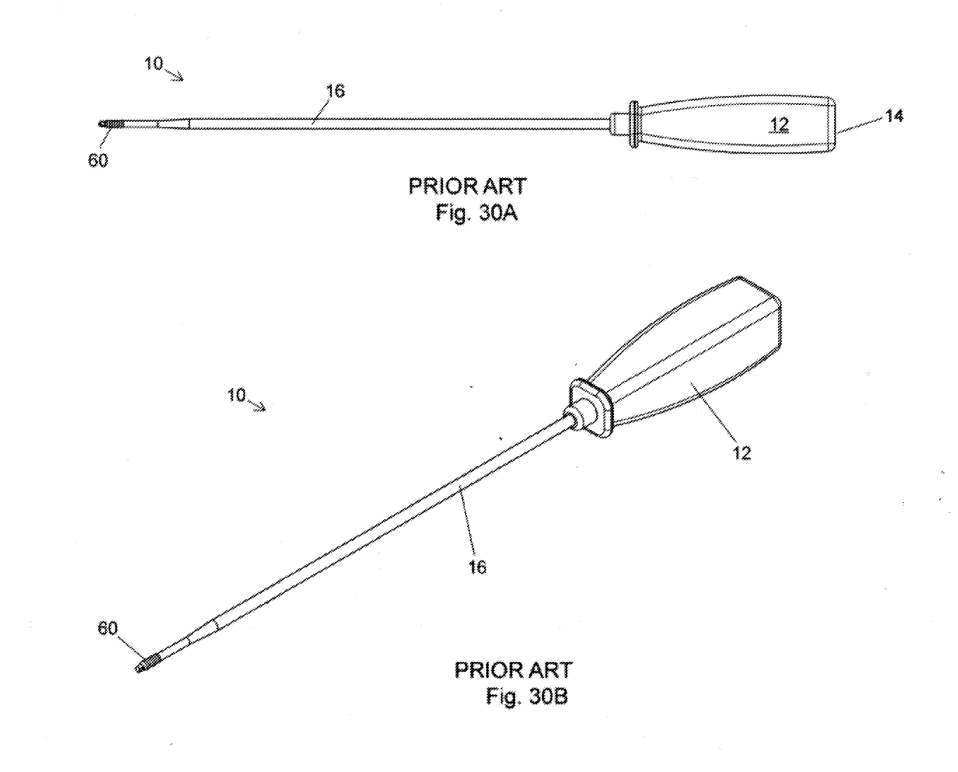

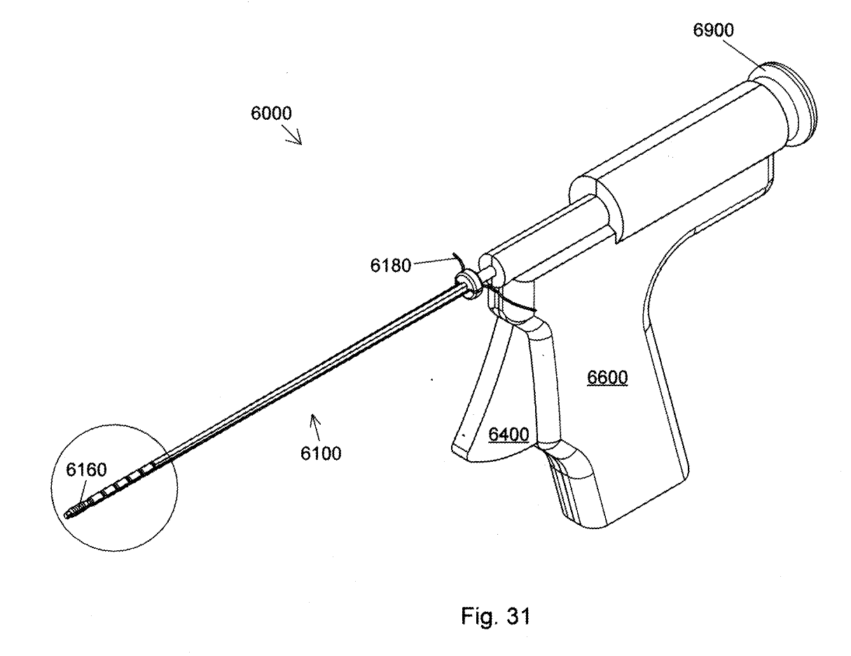

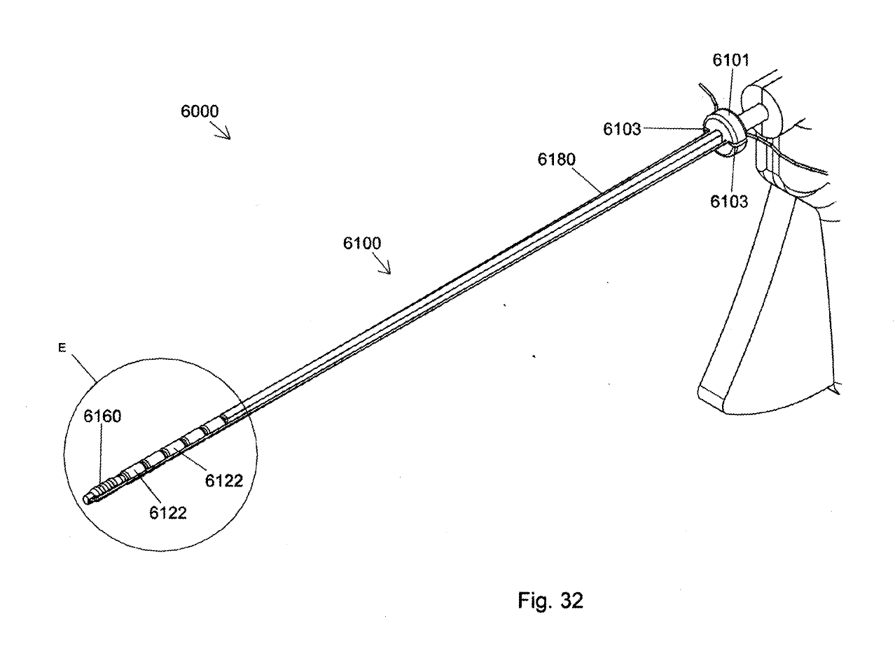

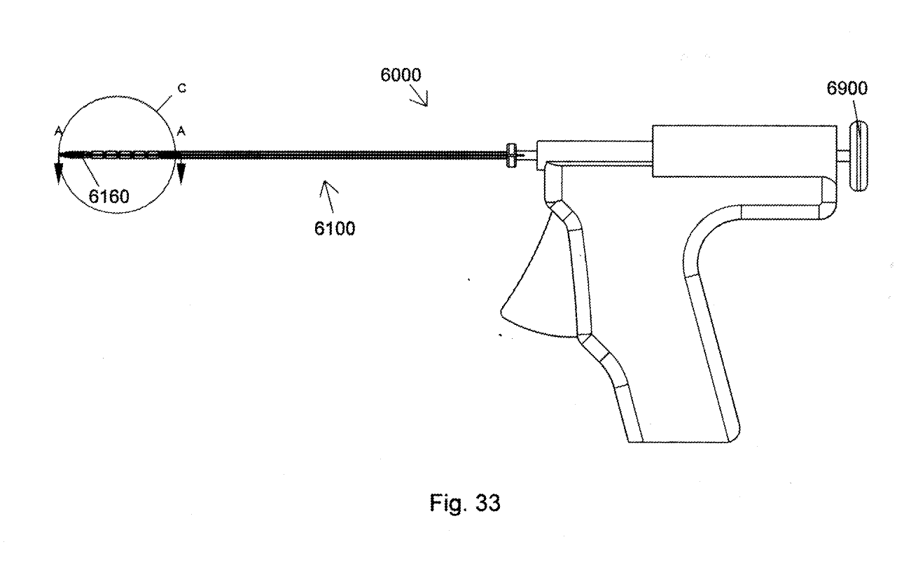

Prior art devices and methods for placing push-in implants and interference-plug type anchors, such as knotless or pre-loaded suture anchors, employ an elongate driver device having a proximal handle portion with a proximal-most surface, and an elongate distal portion. In conventional practice, the implant is removably mounted onto the distal end of the elongate distal portion. The implant is then positioned at the opening of a prepared socket and advanced distally into the socket by means of a force applied to the proximal (handle) portion of the driver device. The placement force is typically applied as a series of percussive blows administered by a mallet. Illustrative examples of prior art implant and driver systems are set forth in FIGS. 29A, 29B, 30A and 30B. As depicted therein, driver device 10 has a proximal handle portion 12 and an elongate distal portion 16 whereon is formed distal-most portion 18 configured to engage a complementary recess in implant 60 so that implant 60 may be removably positioned thereon for placement in a prepared socket. FIGS. 30A and 30B depict implant 60 mounted onto driver device 10 in preparation for use. Handle portion 12 of driver device 10 has a proximal-most surface 14. In conventional practice, the implant 60 is driven axially into a prepared socket by means of a secondary percussive force transmitted to implant 60 by the driver 10. The primary percussive force is applied directly to driver device, generally 10 as a series of blows applied to proximal surface 14 of handle portion 12 by a mallet until such time as implant 60 is deemed driven to the proper depth.

When placing a push-in implant or interference-plug type anchor using prior art driver device 10, the surgeon positions implant 60 at the socket with one hand while maintaining direct visualization by positioning the endoscope with the other hand. An assistant then strikes proximal-most surface 14 of handle portion 12 of driver device 10 as instructed by the surgeon. As noted previously, the assistant may often miss proximal surface 14 and strike the surgeon's hand instead, inflicting pain on the surgeon. In addition, the force applied by a mallet strike from an assistant may exceed that desired by the surgeon, resulting in over-insertion of implant 60.

The present invention avoids these problems by providing an implant placement system that may be operated by a surgeon using only one hand. In addition, the implant placement system is designed to automatically deliver consistent, metered amounts of force. In this manner, the surgeon has direct control over placement of the implant through control of the percussive force applied thereto.

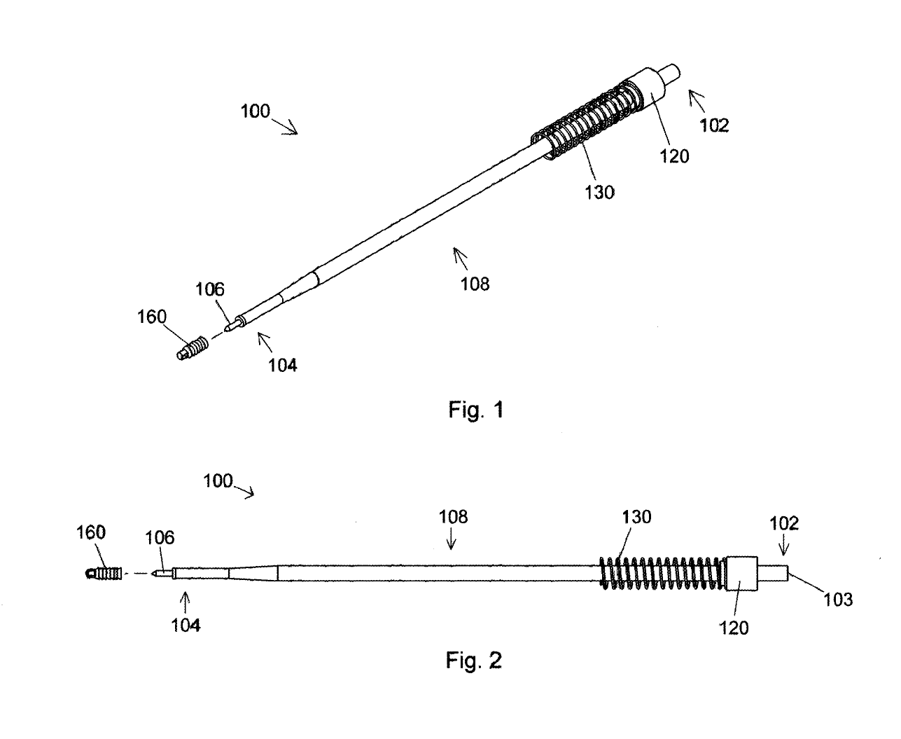

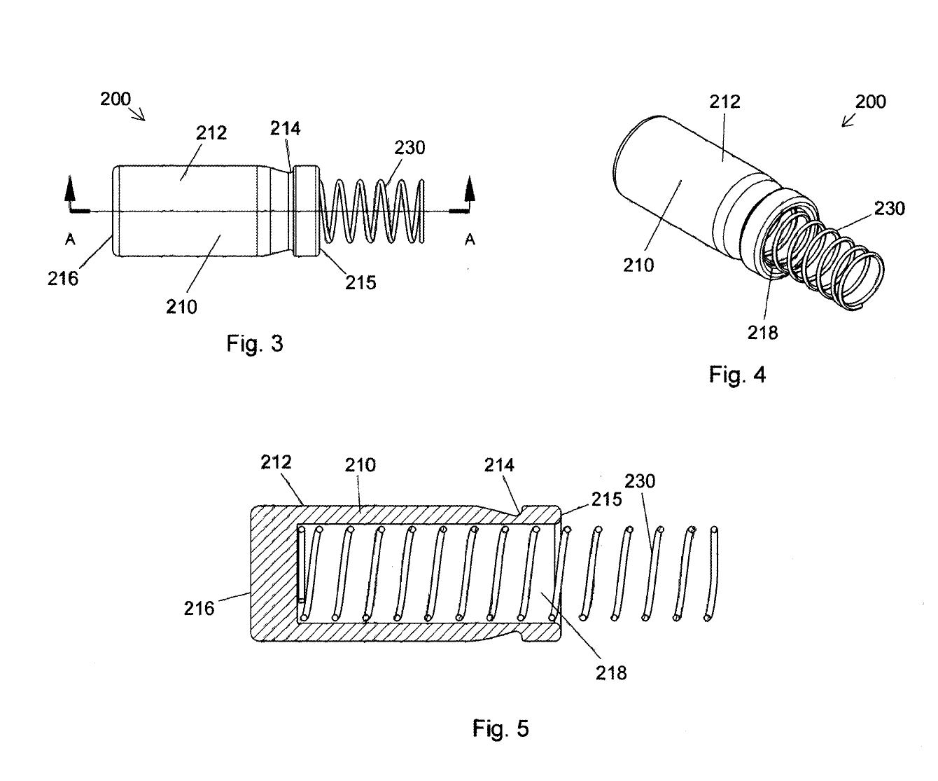

A distal assembly 100 and implant 160 for a hand-held percussive medical device of the present invention is depicted in FIGS. 1 and 2. Elongate metallic element 108 has a proximal end 102 and a distal end 104 wherein is formed distally extending portion 106 configured for removably mounting implant 160. Implant 160 is a push-in style anchor of conventional construction and is not part of the present invention. Cylindrical element 120 is affixed to elongate metallic element 108 a predetermined distance from proximal end 102 and proximal-most surface 103. Spring 130 is positioned on elongate metallic element 108 distal to cylindrical element 120 so as to be able to apply proximal force to assembly 100 when distal assembly 100 is assembled to other elements of the percussive device.

A weight and primary spring assembly 200 for a hand-held percussive medical device of the present invention are depicted in FIGS. 3 through 5. Weight 210 has an outer cylindrical surface 212 wherein is formed groove 214 near the proximal end 215 of weight 210, and a distal-most surface 216. Cylindrical recess 218 is configured to accept therein spring 230.

Linkage assembly 300 has a vertical element 310 with a lower hole 312 configured for pivotably mounting to a fixed element of the percussive device, and an upper hole 316 configured for pivotal mounting to distal end 342 of horizontal element 340 using hinge pin 330. Vertical element 310 has a distal surface 314, and a portion 320 for locating spring 374 that supplies a counterclockwise torque about lower hole 312 to vertical element 310. Horizontal element 340 has a proximal end 344 with beveled distal surface 348 and vertically offset portion 345 with to radius 346 adjacent thereto. Vertically offset portion 345 and radius 346 are configured to engage with groove 214 of weight 210 so that horizontal element supplies a proximal force thereto. Spring 372 is located between protruding portion 318 of vertical element 310 and protruding portion 350 of horizontal element 340 so as to supply a counterclockwise torque about hinge pin 330 to horizontal element 340.

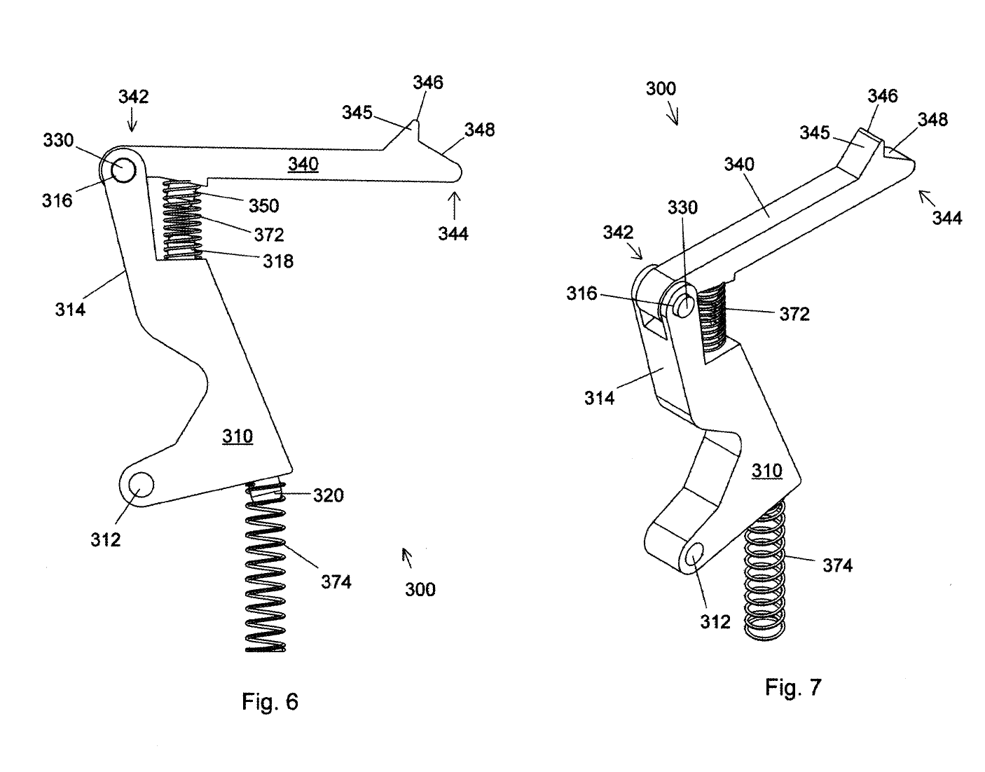

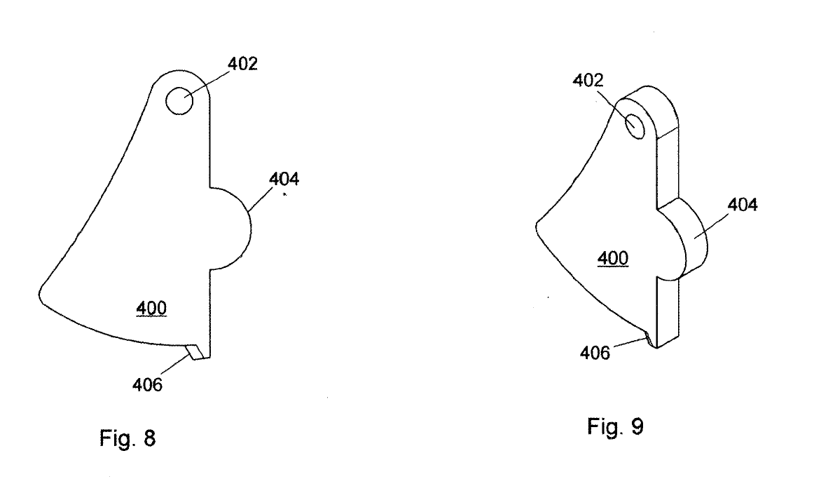

FIGS. 8 and 9 depict an illustrative actuating component, more particularly a trigger element 400 for a percussive medical device of the present invention. Trigger 400 has hole 402 located near its top end, hole 402 configured for rotatably mounting to a fixed element of the percussive device, a radial proximal surface 404 configured for sliding contact with distal surface 314 of vertical element 310 of linkage assembly 300 (FIGS. 6 and 7). Protrusion 406 engages with a fixed element of the percussive device to limit clockwise rotation of trigger 400.

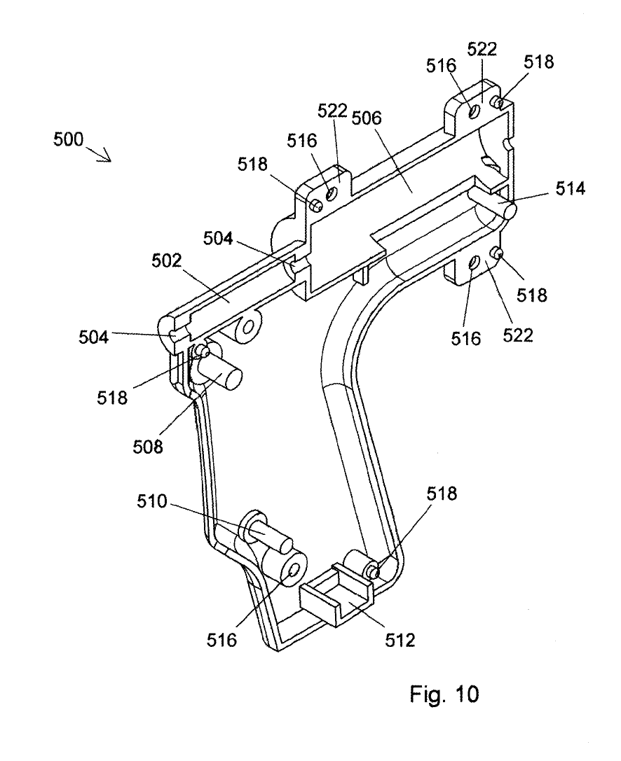

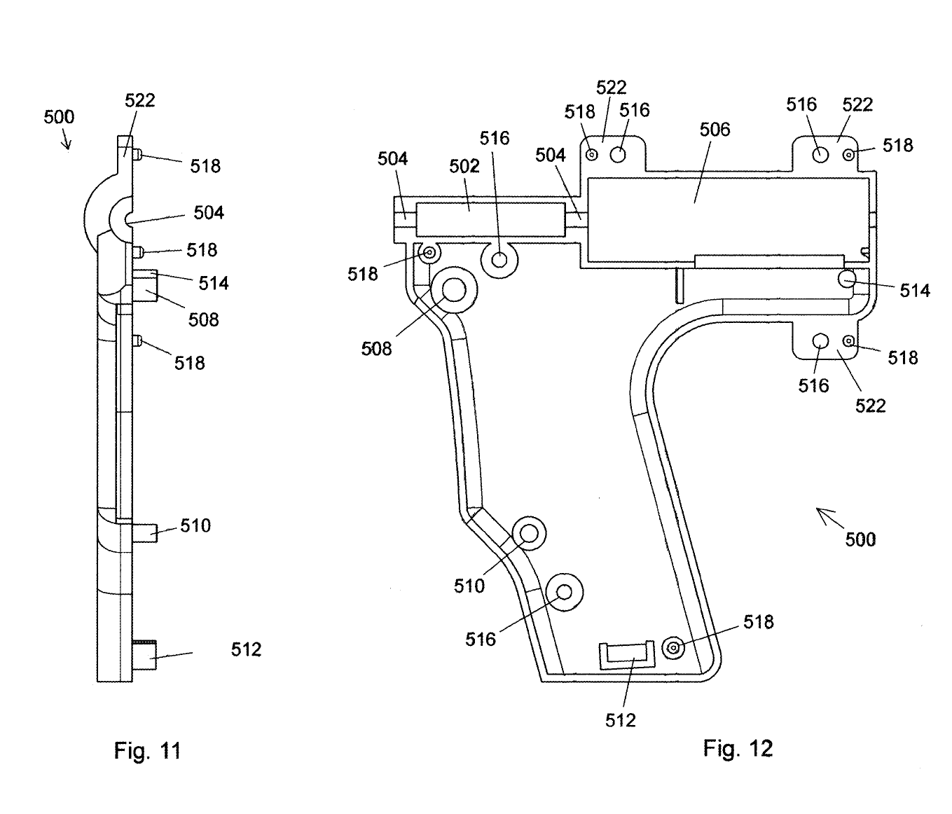

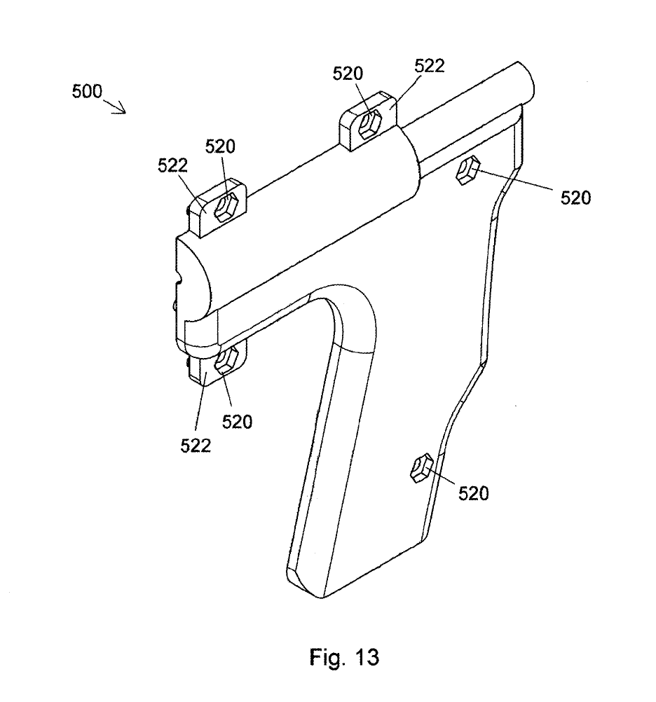

A first case element 500 of a percussive medical device of the present invention is depicted in FIGS. 10 through 13. Case element 500 has distal recesses 502 and 504 configured to receive distal assembly 100. Case element 500 has a proximal recess 506 configured to receive weight and primary spring assembly 200. Protrusions 518 align with complementary features on a second case element for alignment between the two halves that together form a single case assembly. Fastener pairs in holes 516 hold first and second case elements together thereby maintaining the integrity of the assembly. Cylindrical protrusion 508 is positioned and configured to rotatably receive hole 402 of trigger 400 (FIGS. 8 and 9). Cylindrical protrusion 510 is positioned and configured to rotatably receive hole 312 of vertical element 310 of linkage assembly 300 (FIGS. 6 and 7). Cylindrical protrusion 514 is configured and positioned to engage beveled distal surface 348 of horizontal element 340 of linkage assembly 300 (FIGS. 6 and 7). Portion 512 of first case element 500 is positioned and configured to receive the bottom end of spring 374 of linkage assembly 300 (FIGS. 6 and 7). Referring to FIG. 13, hexagonal pockets 520 are configured to receive hex nuts that form half of fastener pairs that maintain the assembly.

Percussive devices of the present invention have two states. In the first state, weight 210 is in a distal position and spring 230 has only an initial compression. In the second state, weight 210 is in a proximal position in which spring 230 is compressed. The transition from the first relatively relaxed state to the second relatively compressed state is accomplished by the surgeon pulling back on trigger 400, the force being transmitted via linkage 300 to weight 200 so as to move weight 200 proximally thereby compressing spring 230. The transition from the second state to the first state occurs when linkage 300 is disengaged from weight 210 and weight 210 is driven distally by spring 230. The potential energy stored in spring 230 is converted to kinetic energy as weight 210 accelerates toward its distal (first position) limit. Prior to reaching this limit, distal-most surface 216 of weight 210 strikes the proximal end of distal assembly 100 thereby transferring the kinetic energy of weight 200 therethrough to implant 160 so as to drive implant 160 a metered incremental distance into a prepared socket. By repeatedly pulling trigger 400 so as to transition the percussive device from its first relatively relaxed state to its second relatively compressed state, and then back to the first, implant 160 is driven incrementally into the prepared socket.

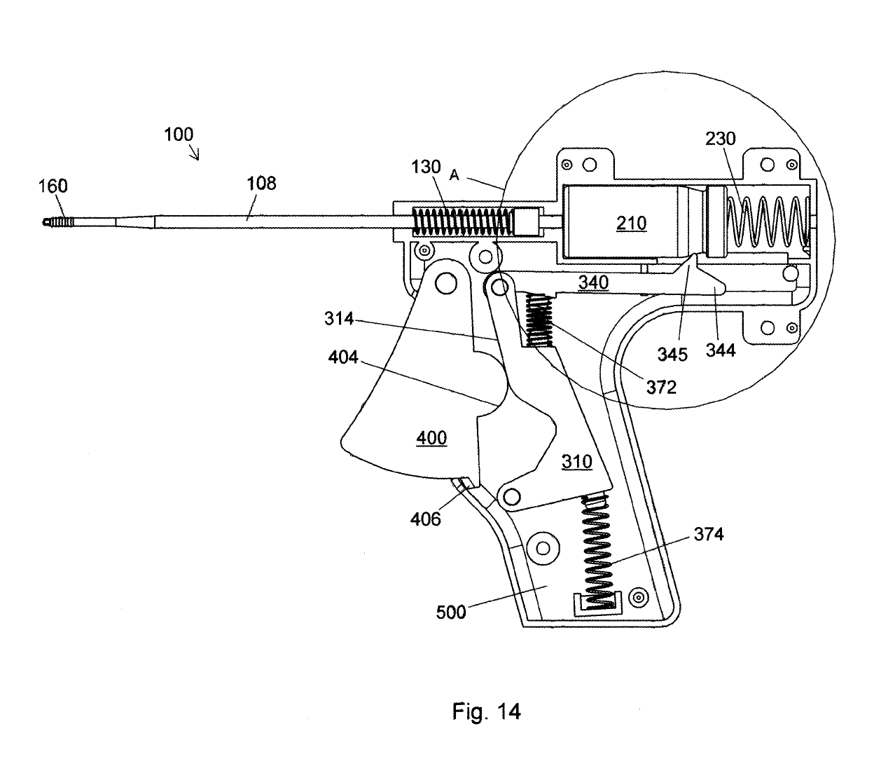

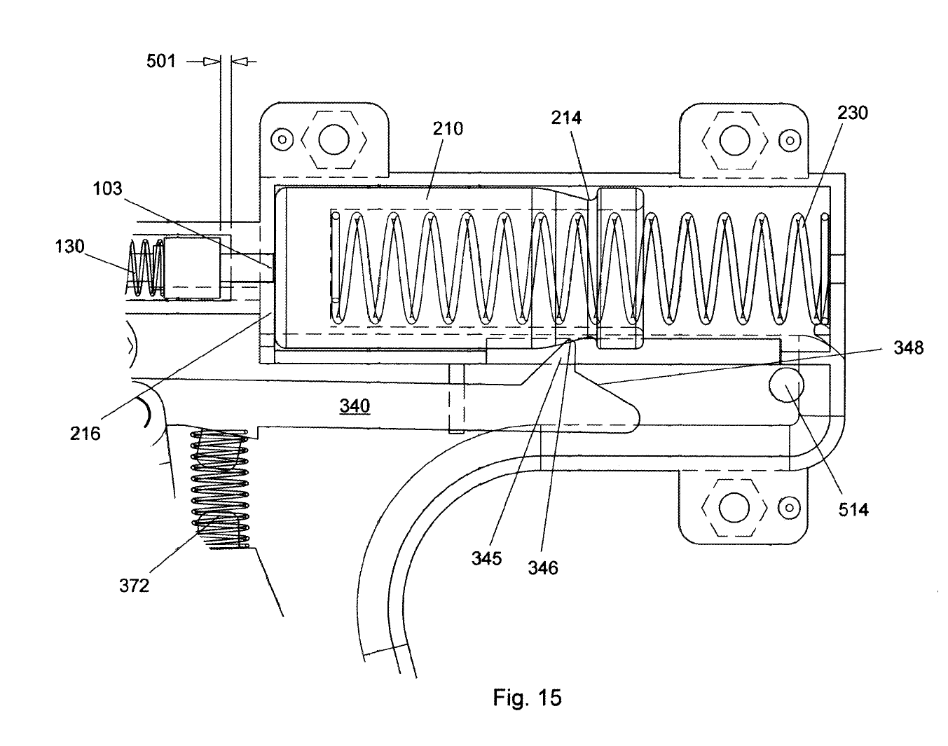

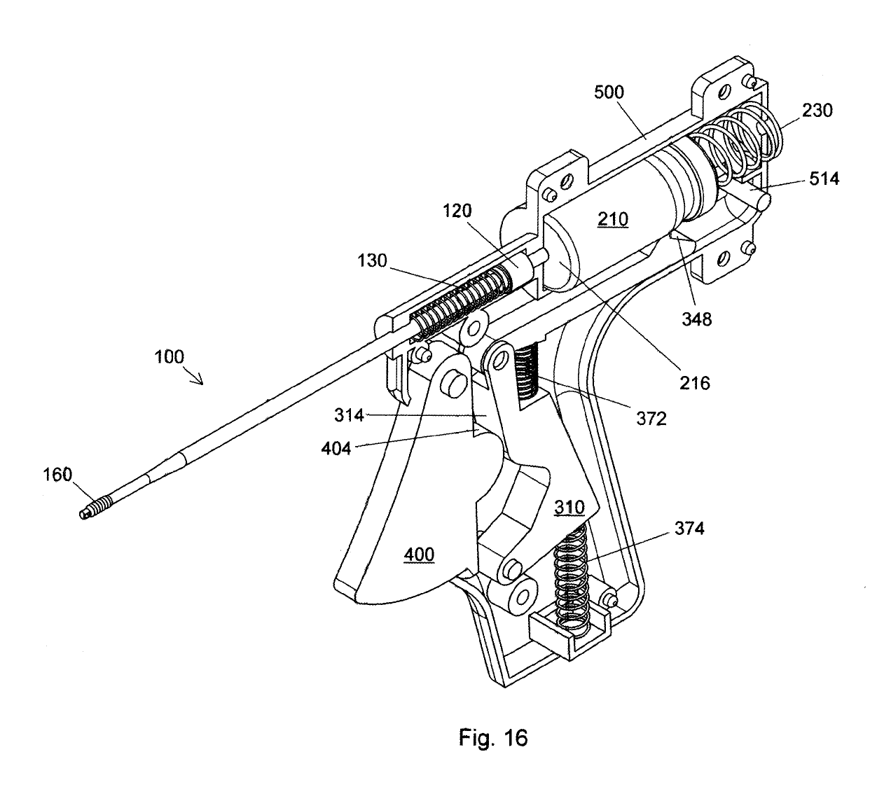

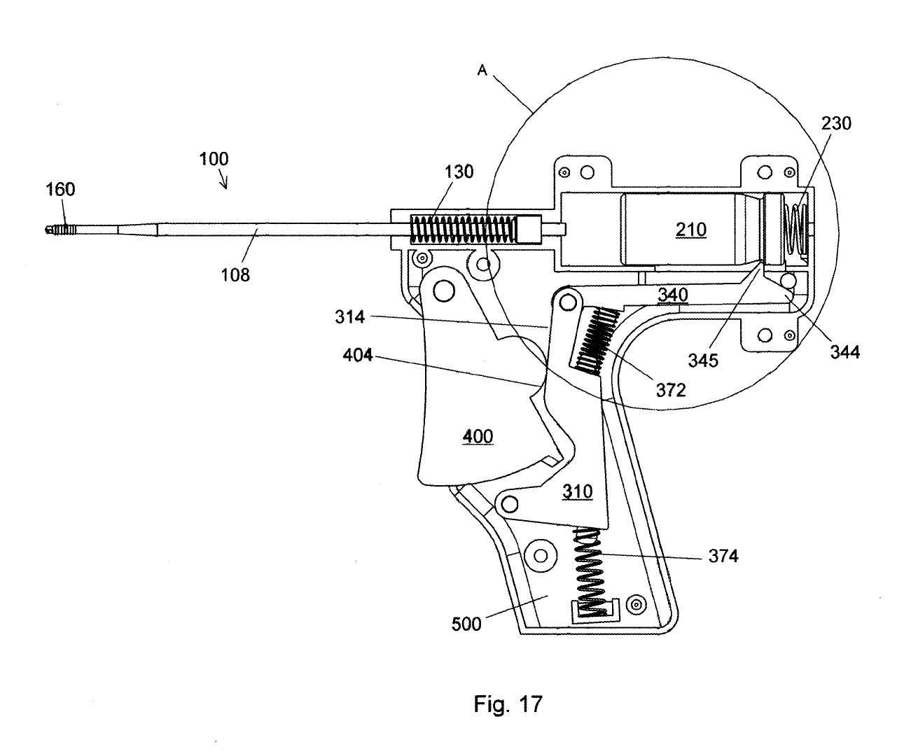

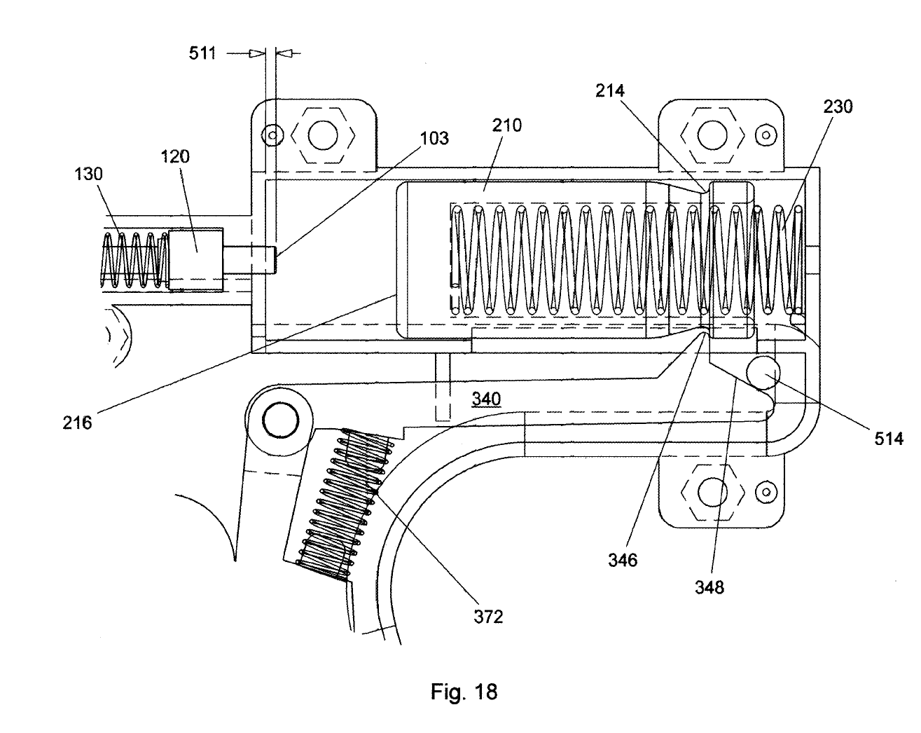

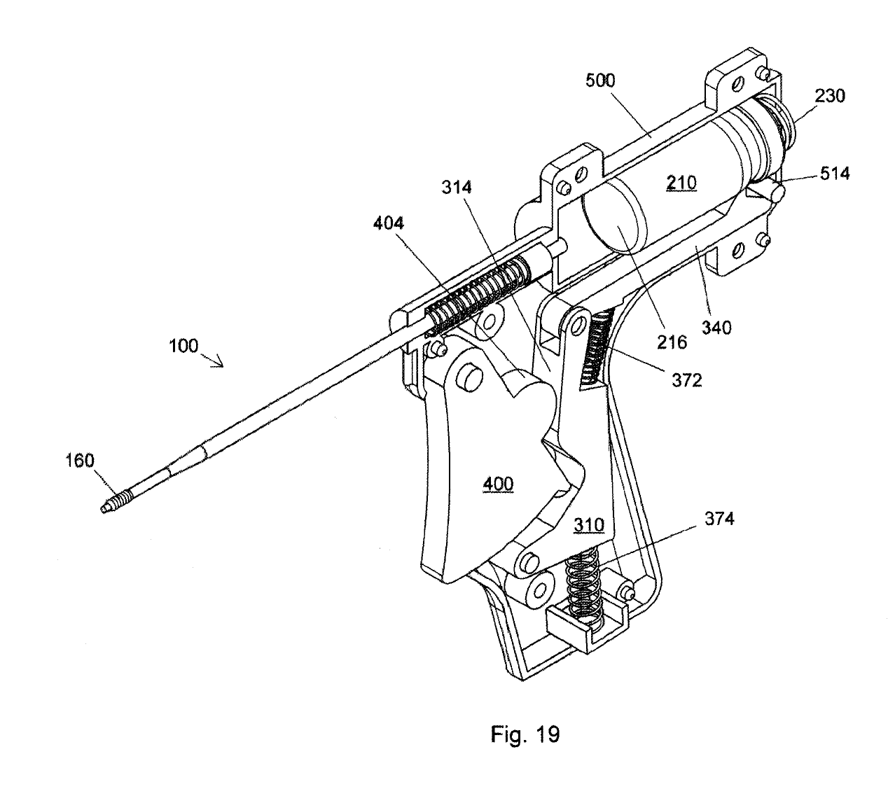

FIGS. 14 through 16 depict first case element 500 with distal assembly 100, weight and primary spring assembly 200, and linkage assembly 300 mounted to first case element 500 as previously described. Elements of the assembly are shown in the first condition, in which the percussive device is ready for use. Protrusion 406 of trigger 400 contacts first case element 500 and is maintained in that position by force supplied by spring 374 to vertical element 310, which transmitted through element 310 to distal surface 314 of element 310, and therethrough to radial proximal surface 404 of trigger 400. Radius 346 of vertically offset portion 345 of horizontal element 340 is maintained in contact with weight 210 by spring 372 such that counterclockwise movement of trigger 400 causes proximal motion of horizontal element 340 resulting in the engagement of groove 214 of weight 210 and vertically offset portion 345 of horizontal element 340. Further counterclockwise rotation of trigger 400 causes horizontal element 340 to travel proximally so as to move weight 210 proximally, compressing spring 230 in the process, a process akin to “cocking” the “hammer” of a gun. Horizontal movement of horizontal element 340 and weight 210 continues until beveled surface 348 of horizontal element 340 contacts cylindrical protrusion 514 of first case element 500 as subsequently depicted in FIGS. 17 through 19. Referring now to FIG. 15, in the first condition of the percussive device, spring 130 maintains contact between proximal-most surface 103 of elongate metallic element 108 and distal-most surface 216 of weight 210 such that there is a gap 501 between the proximal surface of cylindrical element 120 and the proximal wall of recess 502 of case element 500. Proximal movement of weight 210 allows elongate metallic element 108 of assembly 100 to travel proximally such that a portion protrudes distance 511 into recess 506 of case element 500 as shown in FIG. 18. Gap 501 is equal in length to distance 511. When weight 210 is disengaged from horizontal element 340 as shown in FIG. 18, spring 230 accelerates weight 210 in the distal direction towards its first position such that distal-most surface 216 of weight 210 strikes proximal-most surface 103 of elongate metallic element 108 of distal assembly 100 thereby driving implant 130 attached thereto a distance into a prepared socket. By repeatedly pulling trigger 400, implant 130 is incrementally advanced into the socket. In percussive devices of the present invention, force applied to trigger or other activating element compresses a spring thereby creating potential energy that is converted to kinetic energy by accelerating a weight, with the weight subsequently striking a distally extending element so as to transfer a pulse of energy thereto.

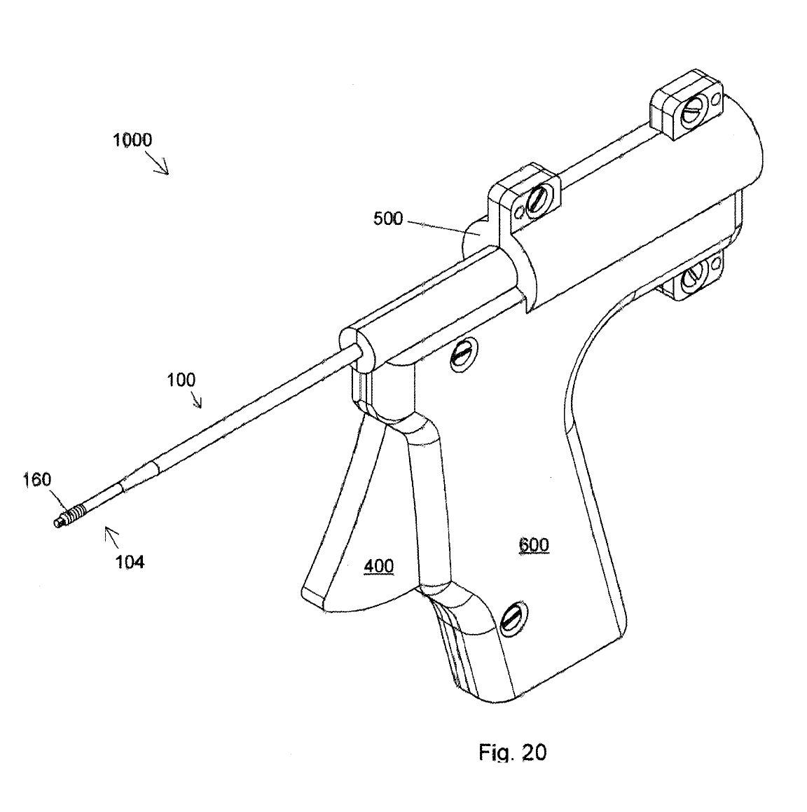

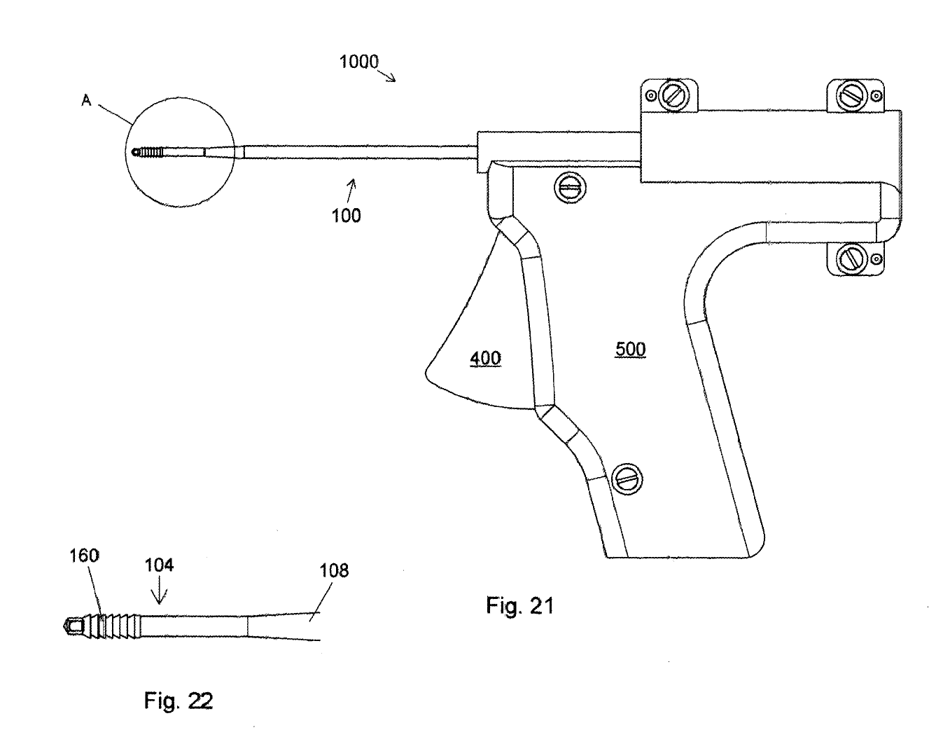

An illustrative example of a completed, fully assembled percussive medical device 1000 of the present invention is depicted in FIGS. 20 through 22. The second case element 600 is affixed to the first case element 500 using fastener pairs. In other embodiments, first and second case elements 500 and 600 are affixed by solvent bonding or ultrasonic welding.

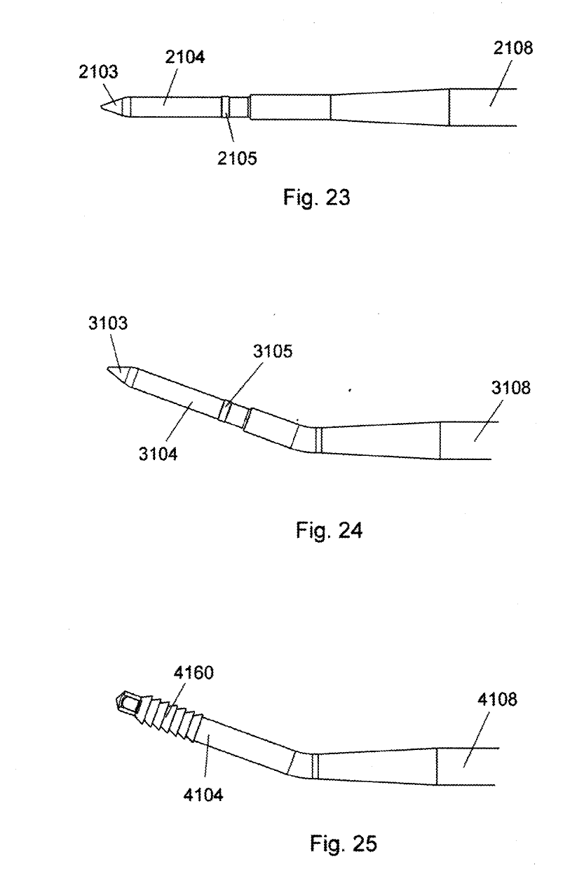

The distal portion of elongate element 108 with implant 160 affixed to distal end 104 thereof is depicted in FIG. 22. In other embodiments, percussive medical devices of the present invention may be used for other purposes. For instance, FIG. 23 shows the distal portion of alternate embodiment device 2000 in which the distal portion is configured for forming sockets in bone as required for implant 160 of FIG. 22. Elongate metallic element 2108 has a distal end 2104 formed to the diameter of the socket required got implant 160, and a sharpened distal end 2103 configured for penetration of bone. Device 2000 replaces the drill or punch conventionally used to form the socket. In a preferred embodiment, distal assemblies 100 and 2100 are removably mounted to the driving device case so that the device may be supplied with both distal assembly 100 and distal assembly 2100. The surgeon may form the socket using distal assembly 2100, then replace distal assembly 2100 with distal assembly 100 on the device and place implant 160. Distal assembly 2100 may be used for other procedures in which holes must be formed in bone. For instance, distal portion 2104 may be formed to a small diameter and used to punch the array of holes required for microfracture treatment of lesions in articular cartilage.

In other embodiments, the distal portion of the distal assembly may be angularly offset as depicted in FIGS. 24 and 25. In form and function, percussive devices 3000 (FIG. 24) and 4000 (FIG. 25) are identical to percussive devices 1000 and 2000 except that sockets may be formed with axes angularly offset from the axis of the device, and implants may be placed in these sockets.

Other embodiments of the present invention are anticipated in which the percussive force may be supplied by the surgeon exclusively as with device 1000 previously herein described, by an assistant with a mallet as in prior art devices, or in a combination of the two methods. For instance, initial insertion of the implant in the socket may be accomplished with force supplied by one or more blows from a mallet, with the balance of the placement using percussive force supplied by the placement device and directly controlled by the surgeon.

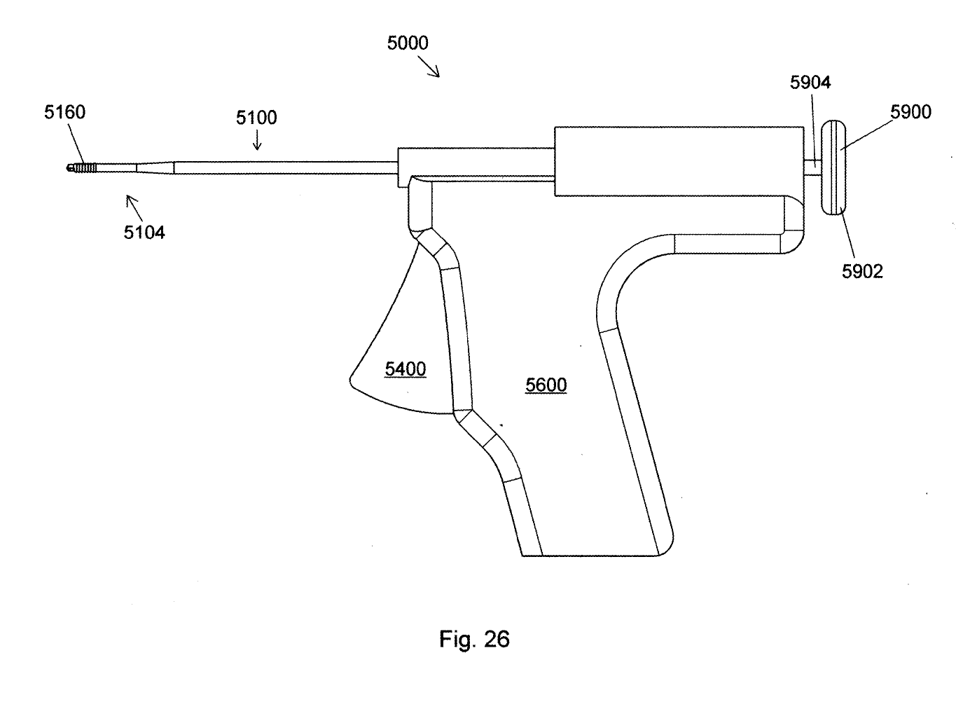

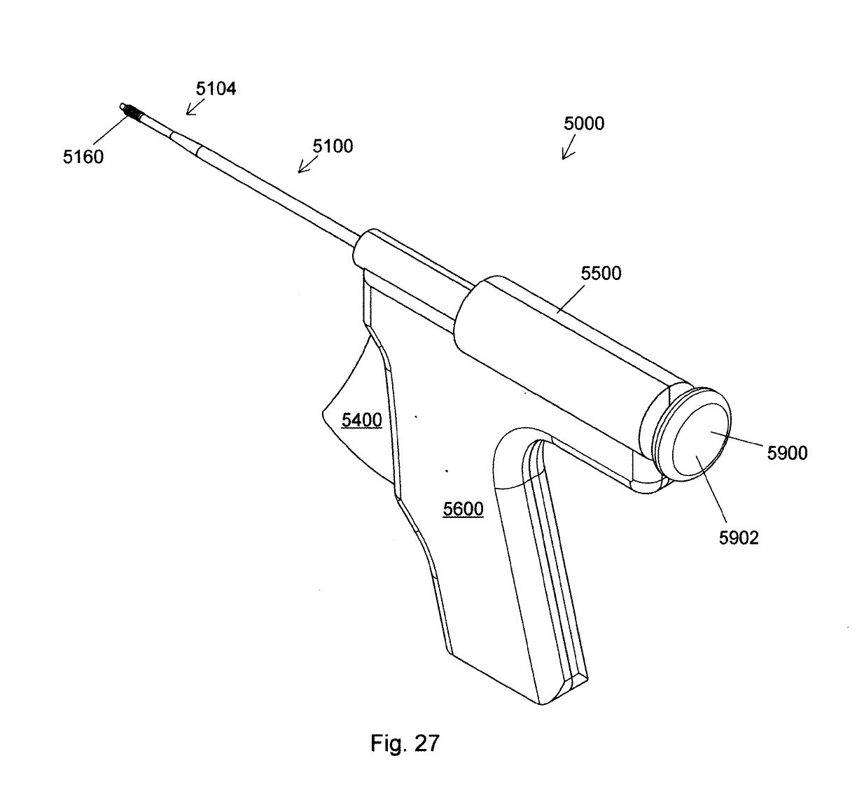

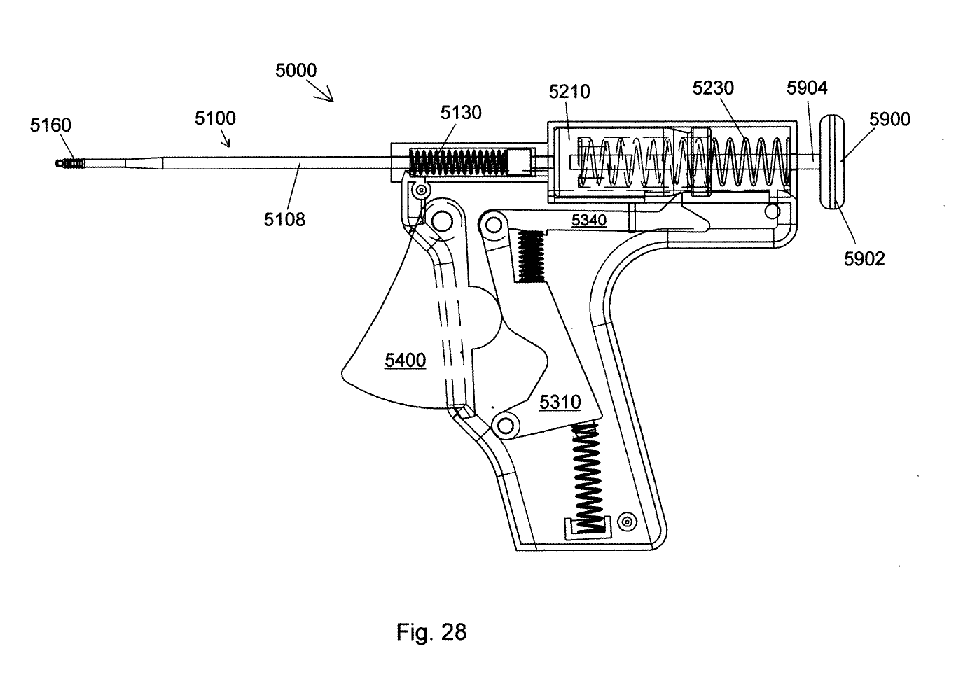

Referring now to FIGS. 26 through 28 depicting alternative “dual mode” embodiment device 5000 of the present invention, device 5000 is like device 1000 in all aspects of form and function except as specifically hereafter described. As with the single-mode device 1000, first and second case elements 5500 and 5600 of device 5000 may be joined by solvent bonding, a suitable adhesive or ultrasonic welding. However, dual mode device 5000 further includes a proximal element 5900 having a proximal portion 5902 and an elongate distal portion 5904 that is affixed at its distal end to weight 5210. The proximal portion of elongate distal portion 5904 of proximal element 5900 with proximally portion 5902 at its proximal end, slidably protrudes beyond the proximal end of joined first and second case portions 5500 and 5600. Proximal element 5900 moves with weight 5210, and force applied to proximal portion 5902 is transmitted by element 5900 and weight 5210 to distal element 5108 and implant 5160 removably mounted thereto. Accordingly, percussive driver device 5000 has two modes of operation: a first in which it functions in the same manner as device 1000 previously herein described, and a second in which the implant is positioned at a prepared socket using device 5000, and implant 5160 is inserted into the socket by blows from a mallet applied to proximal portion 5902 of proximal element 5900. Placing implant 5160 in a prepared socket may be accomplished using either mode singly, or a combination mode 1 and mode 2 depending on surgeon preference. For instance, initially when placing an anchor, a mallet blow (second mode) may be used to help the implant penetrate the hard cortical outer bone portion, after which the surgeon may choose to advance the implant to the required depth using repeated trigger pulls (first mode) with the associated increased control of the placement process.

Referring again to FIG. 24 depicting the distal portion of a device 3000 of the present invention, the angle to which distal punch portion 3104 may be offset from elongate element 3108 is limited because the percussive force applied thereto by element 3108 is not parallel to distal punch portion 3104. As the angle is increased, the tendency of the punch portion to skive the boney surface increases. That is, the likelihood that the sharpened distal end 3103 of distal punch portion 3104 will be displaced from the intended location when percussive force is applied via elongate element 3108 is increased, with the sharpened distal end 3103 frequently forming a groove in the boney surface.

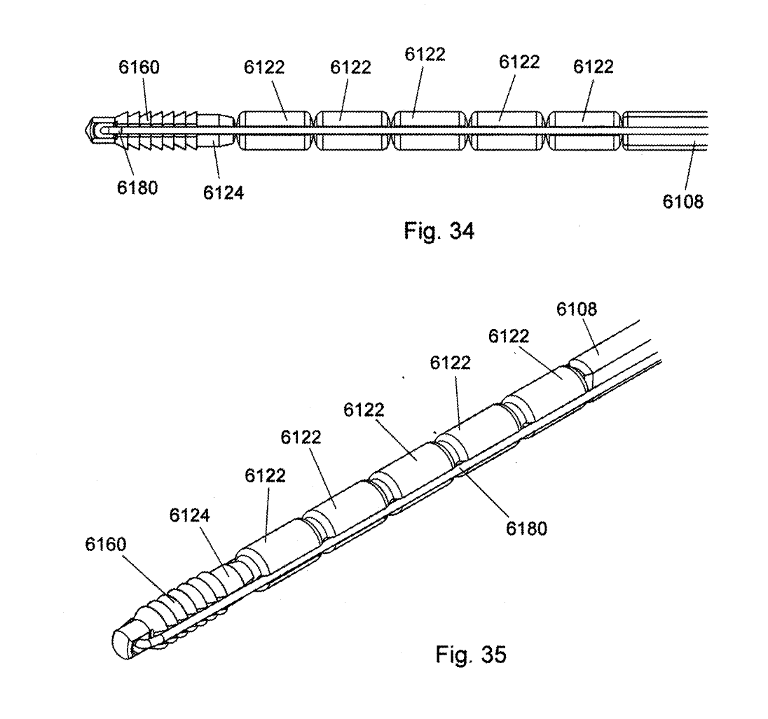

Other embodiments of the present invention allow sockets to be routinely formed in a boney surface and implants placed therein at angular offsets to the percussive device greater than may be achieved with device 3000 of FIG. 34 and device 4000 of FIG. 25. These alternate embodiment devices have a distal portion with a plurality of elements that transmit percussive force. Confining these elements within a tubular external guide allows force to be directed by the guide. In preferred embodiments, the guide has a distal portion that is angularly offset from the proximal portion so that percussive force may be transmitted to an angularly offset distal element for the purpose of forming a socket in a boney surface and for placing an implant in a socket so formed.

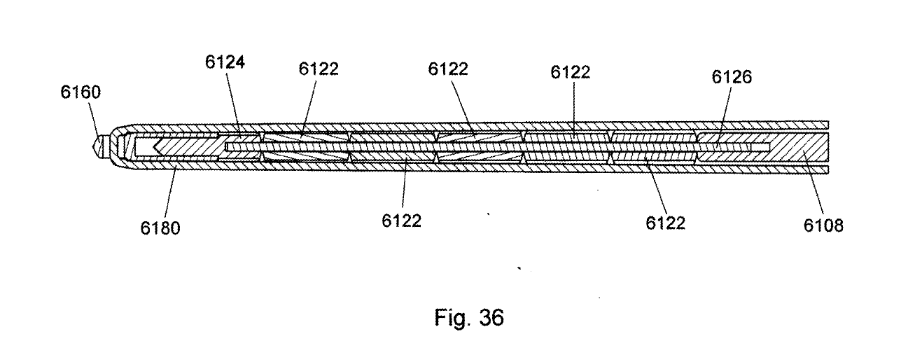

Alternate embodiment device 6000 of the present invention is identical to device 5000 (FIGS. 26 through 28) in all aspects of form and function except as specifically hereafter described. Namely, instead of the unitary elongate metallic element 5108, assembly 6100 has a distal assembly formed of a plurality of discrete metallic elements 6122 leading to a distal-most element 6124 on which anchor 6160 is positioned. As best seen in FIG. 36, flexible elongate element 6126 extends from the distal end of elongate element 6108 to which it is affixed, through links 6122, to distal-most element 6124 to which it is affixed. In a preferred embodiment, flexible element 6126 is formed of a braided metallic material such as, for instance, braided stainless steel. Fixation to elongate element 6108 and to distal-most element 6124 of flexible element 6126 may be by welding, by brazing, or by mechanical means, the fixation means having sufficient strength to prevent failure from tensile forces created during implant placement and the subsequent withdrawal of device 6000. Distal element 6124 and elements 6122 have formed therein laterally opposed, axially extending slots to accommodate suture 6180 that extends from implant 6160, proximally along elongate element 6108 on which axially extending flats are formed, to flange 6101 in which suture retaining slots 6103 are formed.

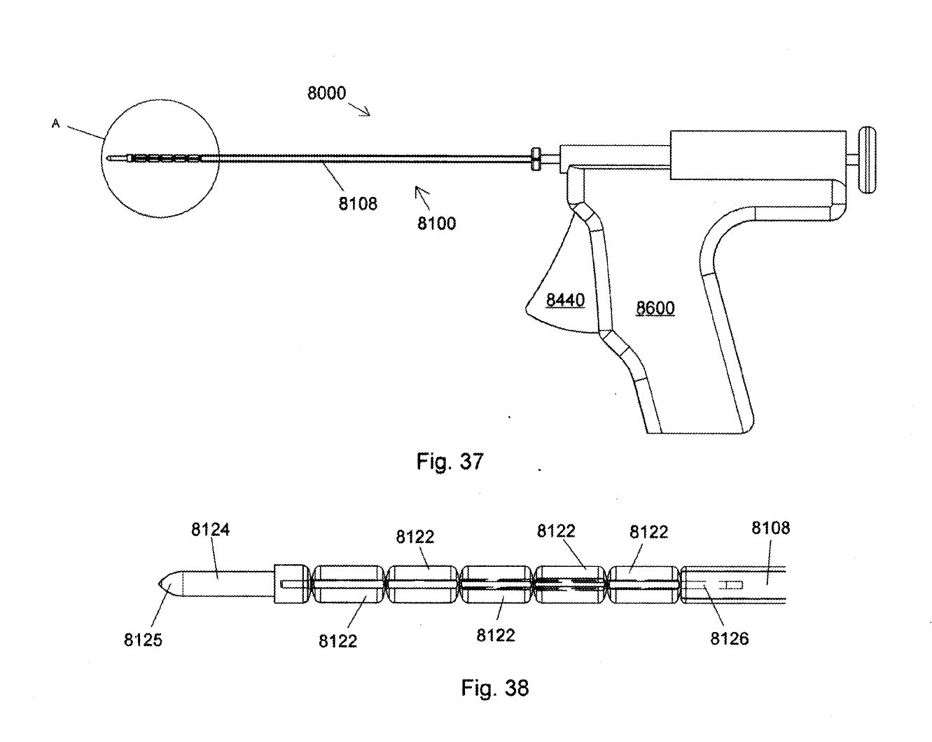

Another alternate embodiment device 8000 of the present invention, configured for the forming (punching) of sockets in a boney surface is shown in FIGS. 37 and 38. Device 8000 is identical to device 6000 in all aspects of form and function except as specifically subsequently described. Namely, distal element 6124 and implant 6160 of device 6000 are replaced by distal element 8124 with sharpened distal end 8125. Distal element 8124 is configured for axially forming a socket in a boney surface when percussive force is applied to its proximal end by elements 8122. Suture 6180 is eliminated.

Forming a socket in a boney surface using device 8000, and placing implant 6160 in that socket can be improved and expedited through use of a tubular guide device, wherein the distal end of the guide is positioned at the predetermined location for placement of implant 6160. Percussive devices 6000 and 8000 may be inserted into the proximal end of the guide.

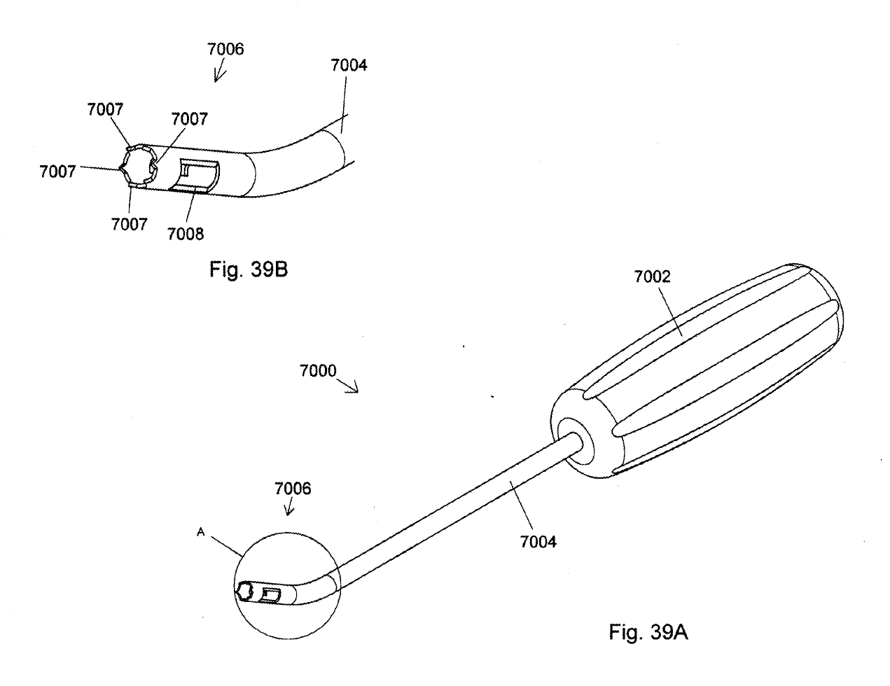

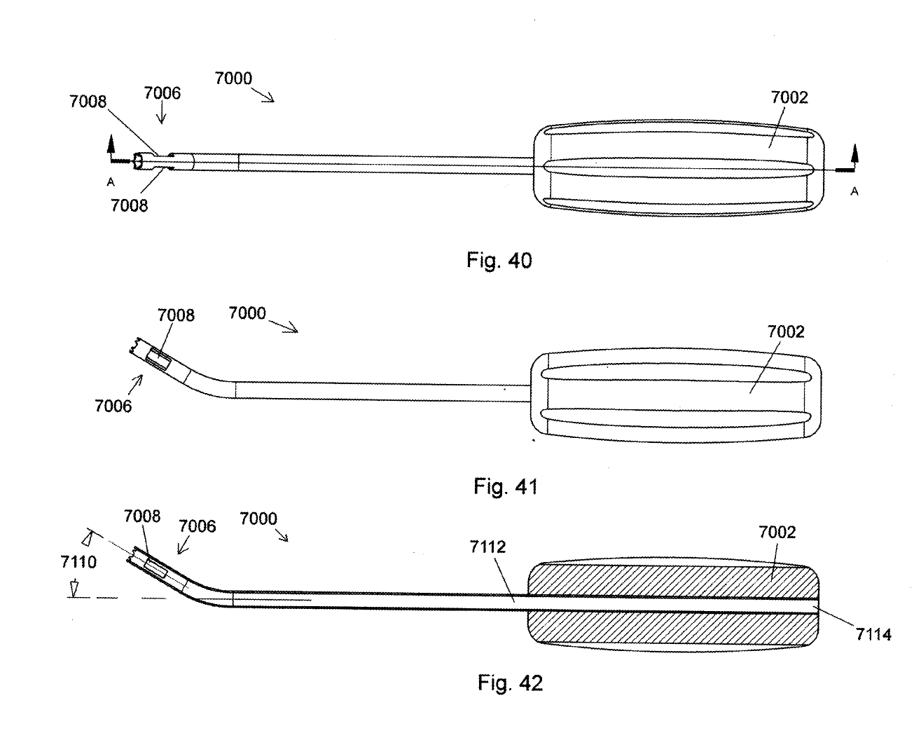

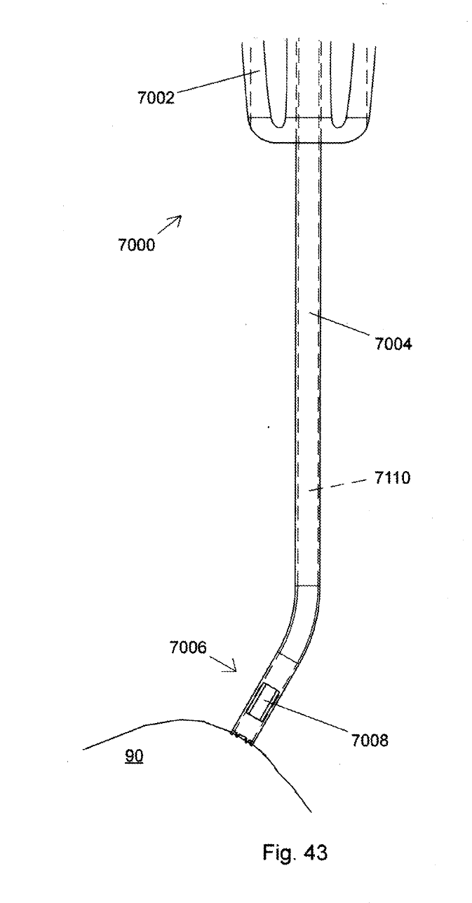

An illustrative guide device 7000, shown in FIGS. 39 through 43, has an elongate tubular element 7004 with a handle 7002 formed on its distal portion and a distal portion 7006 in which are formed openings 7008. Distal portion 7006 is offset angle 7110 from the axis of device 7000. Cannulation 7112 has a proximal opening 7114 into which the distal portions of suitably configured devices may be inserted. Distal portion 7110 may optionally have sharpened anchoring protuberances or “claws” 7007 formed on its distal-most surface, such protuberances configured to penetrate the boney surface when the guide is positioned to prevent movement of the guide distal end during socket formation and implant placement.

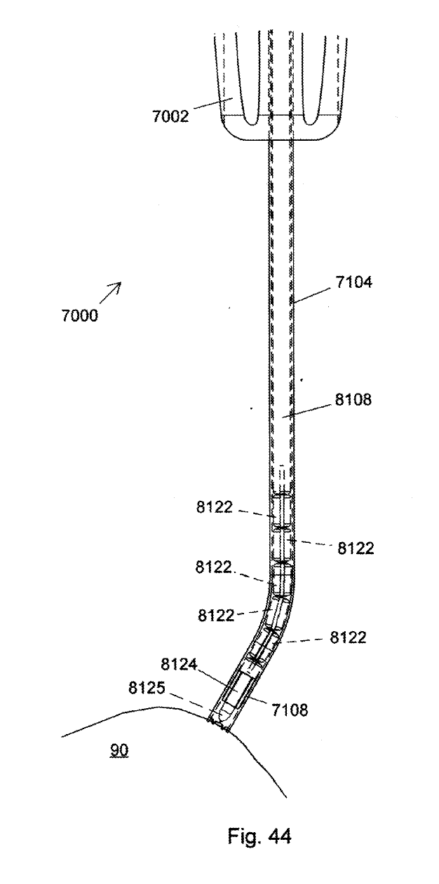

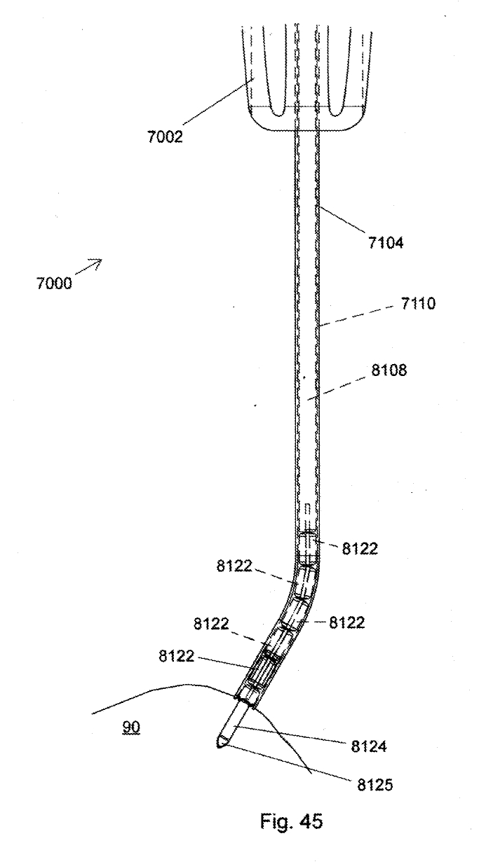

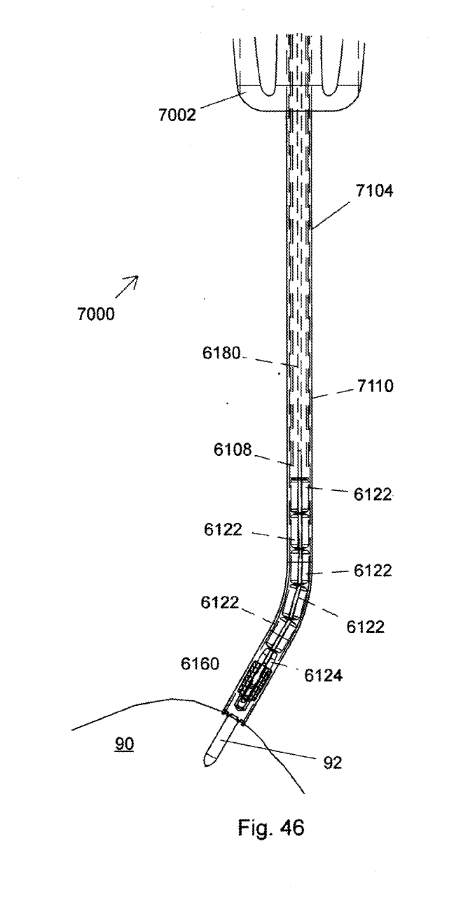

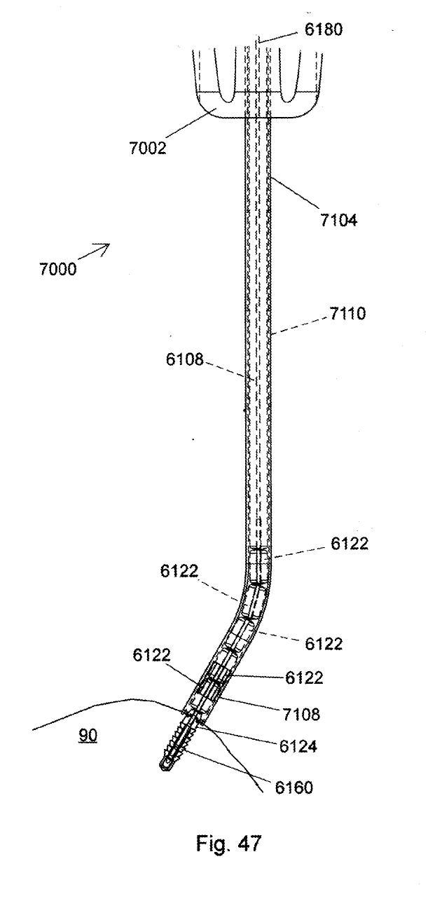

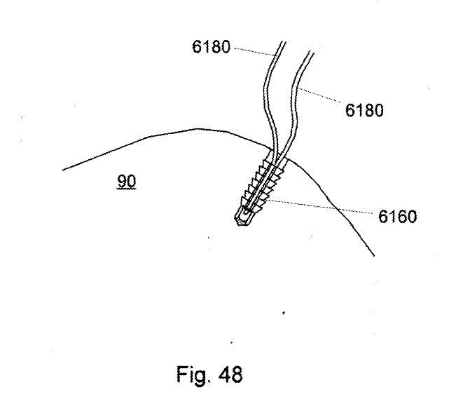

Hereafter, a method of the present invention for placing a push-in implant in a boney surface at a location that cannot be accessed using standard, rigidly linear devices is described. In a first step, guide 7000 is positioned with its distal end at a predetermined location on bone 90 at which a suture anchoring implant is to be placed. Subsequently, as depicted in FIG. 44, the distal portion of device 8000 is inserted into guide 7000 with the sharpened distal end 8125 of distal element 8124 positioned adjacent to the boney surface. Thereafter, while applying sufficient force to guide 7000 to maintain its position in contact with the surface of bone 90, percussive force is applied as previously described herein until distal element 8124 is fully inserted as depicted in FIG. 45. Device 8000 is then withdrawn proximally from guide 7000. Referring now to FIG. 46, after socket 92 is formed in bone 90 at the predetermined location in the previous step, while maintaining the position of guide 7000, the distal portion of device 6000 with implant 6160 is inserted into guide 7000 as shown. The lateral grooves in links 6122 and distal element 6124 together with flats formed on elongate element 6108 form a path for sutures 6180 from implant 6160 to slots 6103 in flange 6101. Thereafter, while applying sufficient force to guide 7000 to maintain its position in contact with the surface of bone 90, percussive force is applied as previously described herein until distal element implant 6160 is fully inserted as depicted in FIG. 47. Device 6000 is then withdrawn from guide 7000. FIG. 48 depicts implant 6160 placed in bone 90 using a method of the present invention.

In the method previously described, socket 92 is formed in bone 90 using a percussive device 8000 of the present invention. In an alternate method of the present invention, the socket may be formed by a drill configured for use with guide 7000.

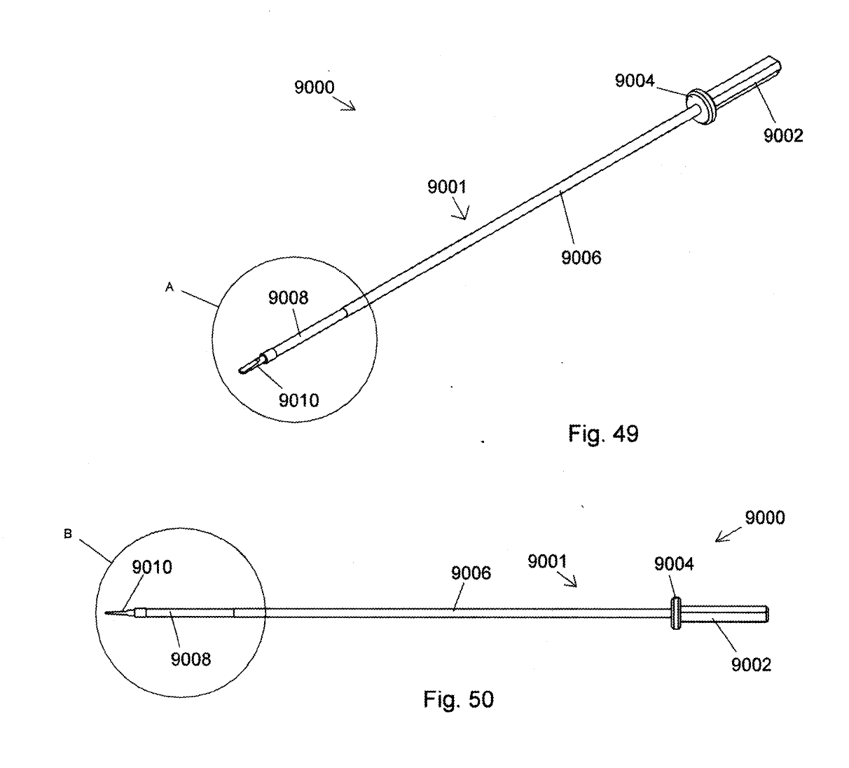

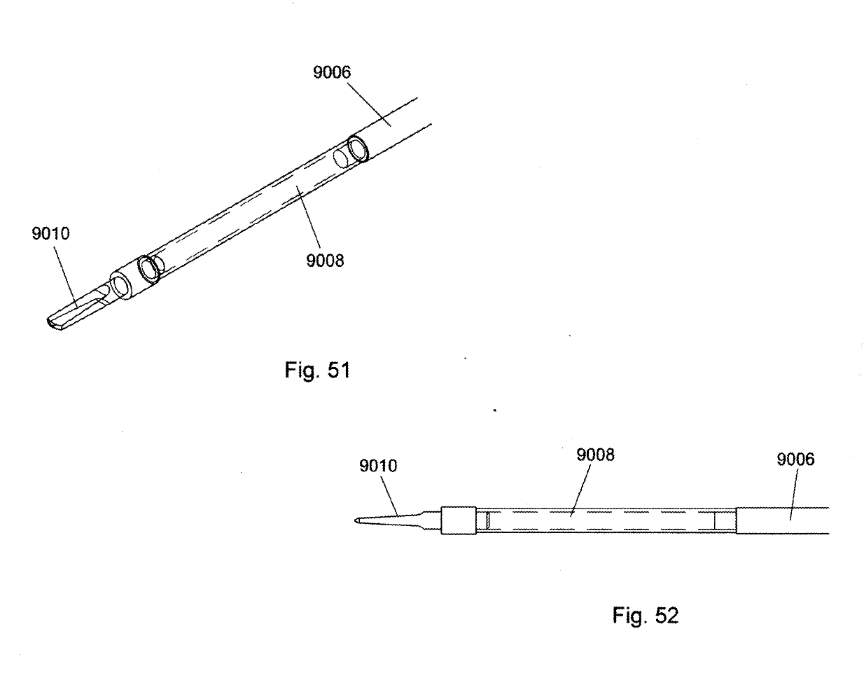

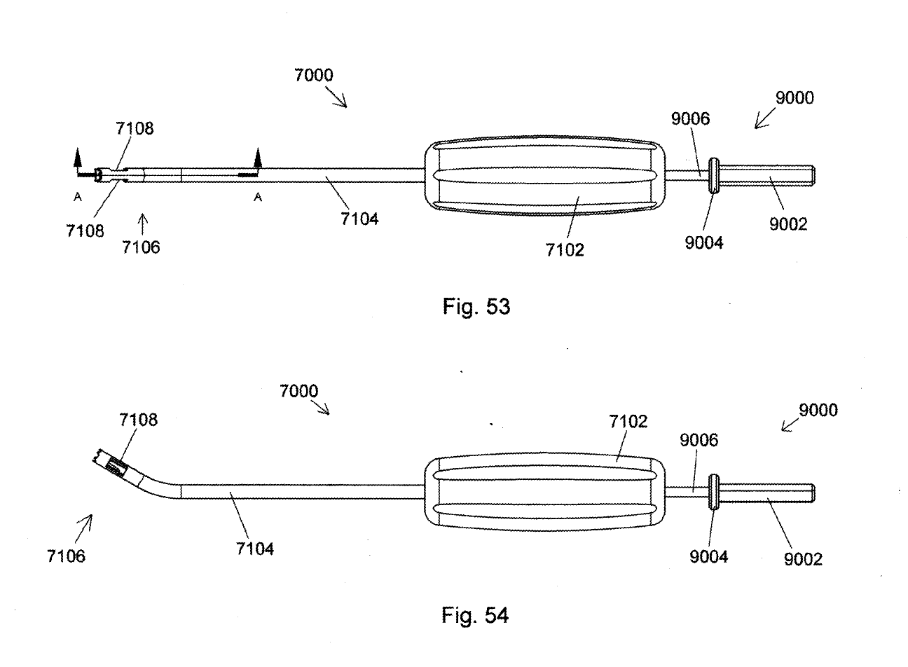

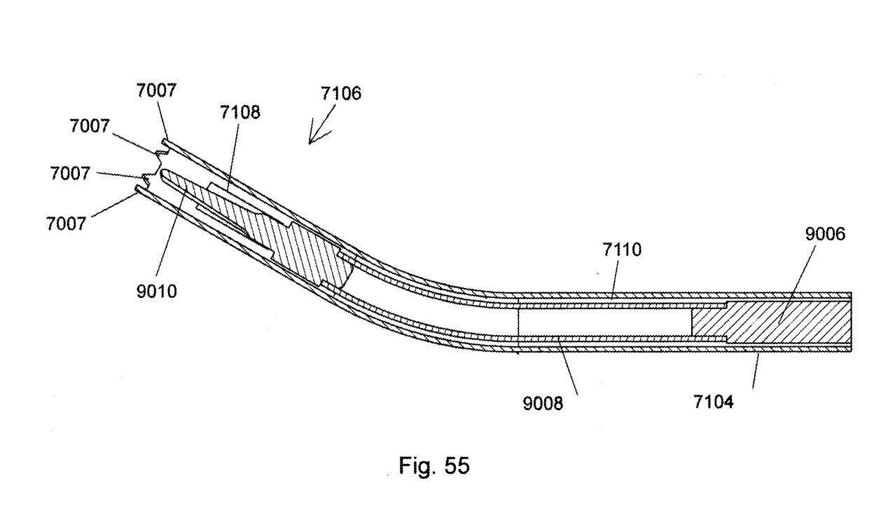

An illustrative drill 9000, depicted in FIGS. 49 through 52, takes the form of an assembly formed of an elongate proximal element 9001, a flexible torque-transmitting middle element 9008, and a distal element 9010 configured for drilling a socket in a boney surface. Elongate proximal element 9001 has a proximal portion configured for removably mounting in a powered handpiece, and an elongate distal portion 9006. Flange 9004 is positioned between proximal portion 9002 and elongate distal portion 9006. Drill 9000 is sized and configured for insertion into guide 7000 and rotational and axial motion when fully inserted therein. FIGS. 53 through 55 depict drill 9000 positioned in guide 7000 as during use.

In methods of implant placement of the present invention in which the insertion site is not accessible using rigidly linear devices, drill 9000 may be substituted for percussive devices of the present invention for forming a socket without departing from the principles of the invention.

Previously described herein is a single mode device 1000 wherein the percussive force originates within the device, and dual mode devices 5000, 6000 and 8000 wherein the percussive force may originate within the device or may be supplied (or amplified) by an external means such as a mallet and simply transmitted through the device. In other single mode embodiments of the present invention, only externally generated percussive force is used, the devices only serving to transfer percussive force from the external source to the device distal element. Unlike prior art drivers for push-in implants, the elongate driving element need not be in a fixed axial position relative to the handle, but rather is movable within predetermined limits such that percussive force applied to the elongate driving element is not transferred to the handle. In this manner unintended lateral movement of the handle by the surgeon during anchor placement is minimized, along with shocking of the surgeon's hand.

Hand-held percussive devices of the present invention may be used for other tasks in which a surgeon must impart percussive energy, directly or indirectly, to a device or to a boney surface or prefabricated socket. One such exemplary application is the dilation of tibial and femoral tunnels formed by the surgeon when repairing cruciate ligaments in a knee. It is conventional in the art for the surgeon to drill each tunnel in a predetermined location. In certain circumstances, it is desirable to dilate the tunnels after they are formed. That is, a cylindrical device slightly larger than the diameter of the drilled tunnel is forced into the tunnel so as to compress bone surrounding the tunnel. A series of dilators may be introduced into the tunnel or socket, each slightly larger than the previous to achieve optimal compaction of the surrounding bone. Conventionally, such dilators are driven in using a mallet, a procedure that, as discussed above, requires a second set of hands and is associated with incomplete and/or uncontrolled axial movement as well as risk to the surgeon's hands. However, again as noted above, these problems may be avoided through the use of a hand-held percussive device of the present invention to drive these devices.

When a bone-tendon-bone patellar graft is used for repair of a torn ACL, the graft has a bone plug on each end of the tendon. When the graft is harvested from the patient, the harvesting procedure produces a graft with bone plugs that have a square cross-section. However, before the graft can be placed in a knee with conventional round drilled tunnels, the bone plugs must be modified to have a cylindrical shape. This modification adds to the procedure time. The requirement for modifying the shape of the square bone plugs to fit a cylindrical tunnel is eliminated if the initial tunnel shape is square (or rectangular or polygonal) and matches the size and shape of the bone plugs. This may be achieved by dilating the cylindrical drilled tunnel with a dilating device with a squared profile so as to produce a tunnel with a matching square cross-section.

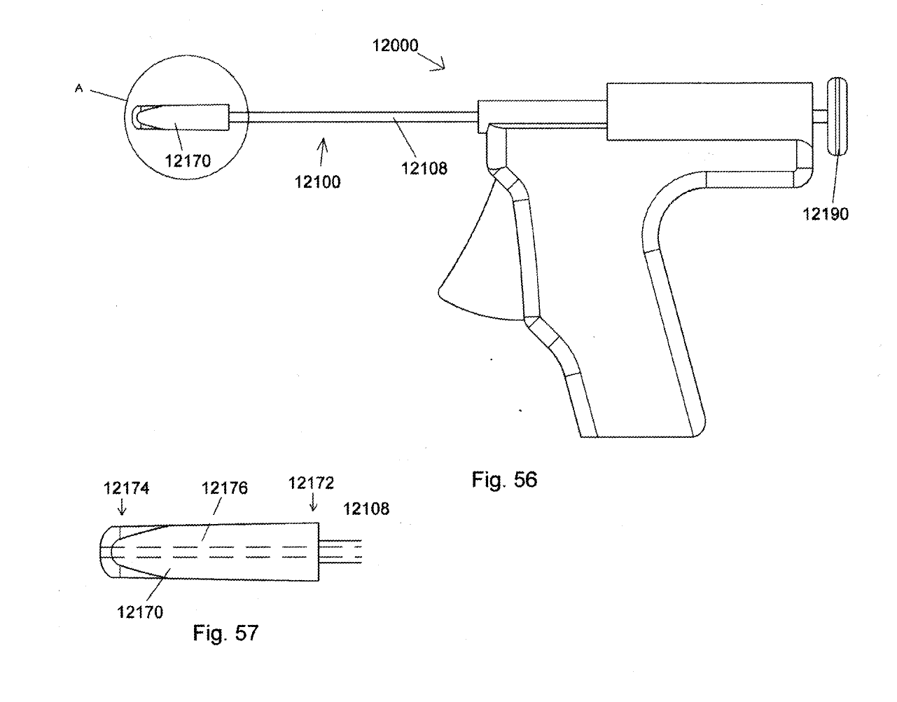

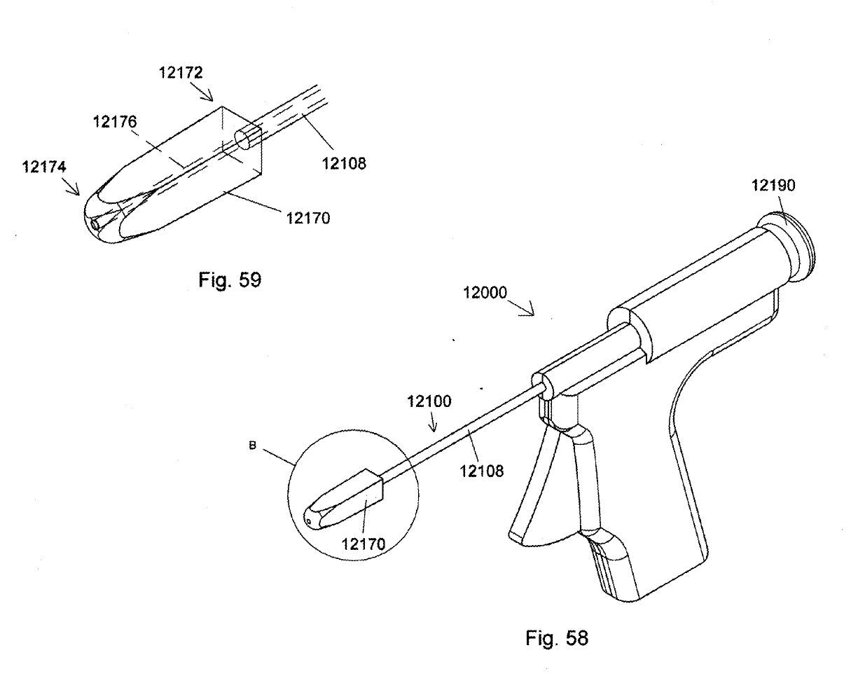

FIGS. 56 through 59 depict a percussive device 12000 of the present invention configured for dilating a cylindrical drilled tunnel to produce a square tunnel. Device 12000 is identical to device 5000 in all aspects of form and function except as specifically described hereafter. Elongate member 12108 of distal assembly 12000 has formed at its distal end dilating element 12170, the proximal end 12172 thereof having a square cross-section and the distal end 12174 thereof having a tapered bullet shape easily insertable into a cylindrical tunnel. Cannulation or lumen 12176 is configured to receive a guidewire therein to aid in alignment of dilating element 12170 with the previously drilled tunnel.

Device 12000 is used in the following manner. A guidewire is placed in the location in which the tunnel is to be created. A cylindrical tunnel is formed, and the drilling device removed. Thereafter, dilating member 12170 of device 12000 is brought to the proximal end of the tunnel, the previously placed guidewire and cannulation 12176 cooperatively aligning member 12170 with the tunnel. While applying distal force to the handle portion of percussive device 12000, percussive force is applied. This percussive force may originate within device 12000, through trigger pulls by the surgeon (a first mode), or by mallet strikes on proximal element 12190 (a second mode). Initial dilation of dense cortical bone may require external mallet blows. After initial dilation has occurred using the second mode, the surgeon may complete the dilation using the first mode, that is, with percussive force generated within device 12000 through trigger pulls.

While the dilating of tunnels has been described using a dual-mode device 12000, it will be understood that any of the single-mode devices previously herein described could be used as well if configured with squared dilating element 12170.

### INDUSTRIAL APPLICABILITY

As noted previously, there is a need in the art for improvement in the tissue grafting arts, particularly in connection with the formation of bone sockets and the placement of plug-type implants and bone grafts. The present invention addresses this need by providing single- and dual-mode hand-held percussive medical devices that allow for controlled socket formation and incremental implant placement. The present invention also provides devices, kits and systems that enable off-axis delivery and polygonal socket formation. Although described in detail with respect to tendon and ligament surgeries, such as in connection with ACL reconstruction, it will be readily apparent to the skilled artisan that the utility of the present invention extends to other graft and joint procedures, such as shoulder and ankle repair.

The disclosure of each publication, patent or patent application mentioned in this specification is specifically incorporated by reference herein in its entirety. However, nothing herein is to be construed as an admission that the invention is not entitled to antedate such disclosure by virtue of prior invention.

The invention has been illustrated by reference to specific examples and preferred embodiments. However, it should be understood that the invention is intended not to be limited by the foregoing description, but to be defined by the appended claims and their equivalents.

What is claimed:

1. A hand-held percussive device for automatically delivering repeated metered pulses of pre-determined axial force to a distal end of an elongate driver assembly that defines the longitudinal axis of the device, wherein said device comprises:
a. a housing having:
i. a longitudinally disposed distal channel configured to slidably but securely receive proximal portion of said elongate driver assembly that includes a proximal hub, wherein said proximal hub can move axially within said distal channel from a first retracted position in which said proximal hub is in contact with the proximal end of said distal channel position to a second extended position in which said proximal hub is compressed against the distal end of said distal channel; and
ii. a proximal channel that is collinear with said distal recess, wherein said proximal channel houses (a) a weight axially aligned with said proximal hub of said elongate driver assembly, and (b) a spring axially aligned with and assembled to said weight, wherein said weight-and-spring assembly is characterized by a first state in which said weight is pressed against the distal end of said proximal channel, in contact with the proximal hub of said elongate driver assembly, and said spring is in a relatively relaxed configuration, and a second state in which said weight is proximally displaced from the proximal portion of said elongate driver assembly and said spring is in a relatively compressed configuration; and
b. actuator assembly including:
i. a trigger component; and
ii. a linkage assembly having:
a) a first portion in contact with said trigger component such that movement of said trigger component causes movement of said first portion, and
b) a second portion that is engageable to and disengageable from said weight, such that, when engaged, proximal movement of said second portion causes proximal motion of said weight, and, when disengaged, said weight moves distally independent of said second portion, and
c) a third portion linking said second portion to said first portion, wherein said third portion comprises elongate elements pivotally joined and pivotally mounted to said case so as to transfer movement from said first portion to said second portion;
wherein activation of said trigger element causes said third portion to engage said weight, to move said weight proximally to compress said spring, and to disengage from said weight when said weight has reached said second state thereby causing said weight to accelerate in a distal direction and strike said proximal hub of said elongate driver assembly, axially moving said proximal hub from its first retracted position to its second extended position to thereby automate the delivery of a metered pulse of pre-determined axial force to the distal end of said elongate driver assembly.

2. The hand-held percussive device of claim 1, wherein said distal channel further includes second spring to be disposed about the proximal portion of said elongate driver assembly, adjacent to said proximal hub, further wherein said second spring applies a proximal force to said elongate driver assembly when it is received within said distal channel.

3. The hand-held percussive device of claim 1, wherein said proximal hub is cylindrical and said distal channel comprises a cylindrical surface area.

4. The hand-held percussive device of claim 1, wherein said trigger component is rotationally activated.

5. The hand-held percussive device of claim 1, wherein said trigger component is activated by linear motion.

6. The hand-held percussive device of claim 1, wherein housing comprises an assembly of first and second opposed case faces.

7. The hand-held percussive device of claim 6, wherein said first and second opposed case faces are affixed together by fastening pins.

8. The hand-held percussive device of claim 6, wherein said first and second opposed case faces are affixed together by solvent bonding or ultrasonic welding.

9. The hand-held percussive device of claim 1, wherein a proximal end of said weight is attached to a horizontal shaft that extends in the proximal direction beyond said proximal channel to the exterior of said housing and terminates is a vertically aligned proximal facing strike hub surface, whereby application of an axial force to said hub surface causes said weight to move axially in a distal direction and strike said proximal portion of said elongate driver assembly, axially moving said proximal portion from its first retracted position to its second extended position to thereby deliver a manually applied axial force to the distal end of said elongate driver assembly.

10. The hand-held percussive device of claim 1, wherein said linkage assembly comprises gears or gear segments.

11. The hand-held percussive device of claim 1, wherein said third portion of said linkage assembly comprises a pivotally mounted element in slidable contact with said trigger component.

12. The hand-held percussive device of claim 11, wherein said pivotally mounted element further comprises a hinge portion coupled to a first end of an elongate, linearly translatable element.

13. The hand-held percussive device of claim 12, wherein said elongate, linearly translatable element further comprises coupling means for engaging said weight to enable the application of proximal force thereto.

14. The linkage assembly of claim 1, wherein said linkage assembly further comprises elastic elements.

15. The hand-held percussive device of claim 1, wherein a single activation of said trigger component causes sequentially:
a. engagement of said second portion of said linkage with said weight;
b. axial motion of said weight and compression of said spring,
c. automatic disengagement from said weight when said weight has reached said second state, thereby causing said weight to accelerate in a distal direction and strike said proximal hub of said elongate driver assembly, axially moving said proximal hub from its first retracted position to its second extended position to thereby automate the delivery of a metered pulse of pre-determined axial force to the distal end of said elongate driver assembly.

16. The hand-held percussive device of claim 15, further wherein whereupon releasing said trigger component, said trigger component and said linkage to return to their initial positions.

17. A percussive assembly comprising:
a. the hand-held claim 1 percussive device of claim 1, assembled to
b. an elongate driver assembly comprising a distal end, an elongate body, and a proximal hub, wherein said elongate driver assembly is slidably but securely received within said distal channel.

18. The percussive assembly of claim 17, wherein said distal end comprises a bone-socket-forming punch portion terminating in a sharpened distal tip.

19. The percussive assembly of claim 17, wherein said distal end carries an interference-plug type suture anchor.

20. The percussive assembly of claim 17, wherein said distal end comprises a square or polygonal dilating component for modifying the cross-section of a socket formed in a bone.

21. The percussive assembly of claim 17, wherein said elongate body is made from metal.

22. The percussive assembly of claim 21, wherein said elongate body is rigidly linear.

23. The percussive assembly of claim 21, wherein said elongate body is flexible.

24. The percussive assembly of claim 21, wherein said distal end is angled relative to said longitudinal axis.

25. The percussive assembly of claim 17, wherein a spring is positioned about the proximal portion of said elongate body, adjacent to said proximal hub, further wherein said spring applies a proximal force to elongate driver assembly when it is assembled to said housing, in said distal channel.
